(19) United States
(12) Patent Application Publication
(10) Pub. No.: US 2012/0093783 A1
(43) Pub. Date: Apr. 19, 2012

(54) USE ADIPOSE TISSUE-DERIVED REGENERATIVE CELLS IN THE MODULATION OF INFLAMMATION IN THE PANCREAS AND IN THE KIDNEY

(75) Inventors: Kai Pinkernell, Anna, TX (US); Zheng Feng, La Jolla, CA (US)

(73) Assignee: CYTORI THERAPEUTICS, INC., San Diego, CA (US)

(21) Appl. No.: 13/279,090

(22) Filed: Oct. 21, 2011

Related U.S. Application Data
(63) Continuation of application No. PCT/US2010/032275, filed on Apr. 23, 2010.
(60) Provisional application No. 61/172,152, filed on Apr. 23, 2009.

(51) Int. Cl. A61K 35/12; A61P 1/18; C12N 5/077; A61P 29/00

(52) U.S. Cl. 424/93.7; 435/325

(57) ABSTRACT

Aspects of the present invention relate to the field of medicine, specifically, the effect of adipose tissue and its components on modulating inflammation.

### CROSS-REFERENCE TO RELATED APPLICATIONS

The present application is a continuation of International Patent Application No. PCT/US2010/032275, filed Apr. 23, 2010, by Pinkernell et al., entitled “METHODS OF USING ADIPOSE TISSUE-DERIVED CELLS IN THE MODULATION OF INFLAMMATION,” which claims priority to U.S. Provisional Application Ser. No. 61/172,152, filed on Apr. 23, 2009, by Pinkernell, et al. and entitled “METHODS OF USING ADIPOSE TISSUE-DERIVED CELLS IN THE MODULATION OF INFLAMMATION,” which are hereby expressly incorporated by reference in their entireties.

### FIELD OF THE INVENTION

Aspects of the present invention relate to the field of medicine, specifically, to the study of inflammation and the effect of adipose tissue and its components on modulating inflammation.

### BACKGROUND OF THE INVENTION

Inflammation is the biological response by vascular tissues to harmful stimuli, such as pathogens. The inflammatory response protects the body by removing the harmful stimuli and initiating the healing process. However, inflammation must be a closely regulated process. Left unchecked, inflammation can lead to or intensify numerous diseases such as automimmune diseases, artherosclerosis, renal failure, rheumatoid arthiritis, pancreatitis, etc. For example, acute pancreatitis is a multi-faceted disease that is associated with considerable morbidity and mortality and is associated with inflammation. In the United States alone, more than 300,000 patients are hospitalized annually with pancreatitis. Pancreatitis is a primary factor in about 3,200 deaths, and a contributing factor in about 4,000 additional deaths, annually. Direct costs attributable to pancreatitis top $2 billion annually. See, e.g., Saluja and Bhagat, Gastroenterology, 124(3):844-847 (2003).

The pathologic spectrum of acute pancreatitis ranges from relatively mild edematous to severe hemorrhaging or necrotizing pancreatitis, the latter manifesting itself in pancreatic necrosis. While the milder form of acute pancreatitis results in about 1% mortality, necrotizing pancreatitis, which accounts for about one fourth of the cases, has a mortality rate of between 30 to 50%. Still higher mortality rates occur in when the pancreatitis involves infection. Patients with necrotizing pancreatitis suffer a greater risk of serious pancreatic infection and early death with multi-organ failure.

The timing and type of intervention for patients with acute pancreatitis is controversial. Treatment of the milder forms relies mainly on supportive care. Surgical intervention has not been shown to reduce the mortality rates of sterile (non-infected) acute necrotizing pancreatitis, while infected acute necrotizing pancreatitis is considered uniformly fatal without intervention. In either case, necrosectomy and other aggressive surgical procedures remain the standard of care. (Baron, T. H. and Morgan D. E., “Acute Necrotizing Pancreatitis”, The New Engl. J. Med. 340: 1412-1417 (1999).)

Thus, there is an urgent need for new methods of preventing and/or ameliorating the effects of acute pancreatitis.

### SUMMARY OF THE INVENTION

Aspects of the present invention are directed to the use of adipose derived regenerative cells (e.g., a cell composition that comprises a concentrated population of adipose-derived regenerative cells that comprises stem cells) for the modulation of inflammation, in particular for the treatment or amelioration of pancreatitis or acute kidney injury and/or the amelioration or reduction of a condition associated with these maladies. Accordingly, some embodiments concern a method for reducing an inflammatory response in a mammal in need thereof comprising providing to said mammal an amount of a concentrated population of adipose derived regenerative cells sufficient to reduce the amount of a marker for inflammation in said mammal. By some approaches, the marker for inflammation is IL-3, IL-6, IL-8, IL-12, a Chemokine (C-X-C motif) ligand 2 (CXCL2), or macrophage infiltration. In some approaches, the adipose derived regenerative cells are CD14 positive and/or CD11b positive. In some of these methods, the mammal has a pancreatic disorder or a kidney disorder and/or suffers from a condition associated with a pancreatic disorder (e.g., pancreatitis) or a kidney disorder (acute kidney injury). The adipose-derived regenerative cells (e.g., a cell composition that comprises a concentrated population of adipose-derived regenerative cells that comprises stem cells) can be cultured cells but preferably, they are not cultured and are used after isolation (e.g., freshly isolated cells). That is, aspects of the invention concern the use of a therapeutically effective amount of a concentrated population of adipose derived regenerative cells to prepare a medicament for the reduction of inflammation, wherein said concentrated population of cells is to be administered to a patient in need thereof without culturing the cells before administering them to the patient. As stated above, the adipose derived regenerative that can be used can be CD14 or CD11b positive cells and the mammal can have a pancreatic disorder (e.g., pancreatitis) or a kidney disorder (acute kidney injury). Additional embodiments concern a method of ameliorating pancreatitis or a condition associated therewith comprising selecting a patient that has pancreatitis; administering to said patient a therapeutically effective amount of a concentrated population of adipose derived regenerative cells (e.g., a cell composition that comprises a concentrated population of adipose-derived regenerative cells that comprises stem cells), wherein said concentrated population of cells is administered to said patient without culturing the cells before administration; and optionally, measuring the response of said patient before and/or after receiving said concentrated population of adipose-derived regenerative cells. In some aspects of this method, the patient that has pancreatitis is selected by clinical evaluation by a trained health care professional based on diagnostic approaches and/or observation of symptoms associated with pancreatitis. By some of these approaches, the marker for inflammation is selected from the group consisting of IL-3, IL-6, IL-8, IL-12, a Chemokine (C-X-C motif) ligand 2 (CXCL2), and macrophage infiltration is measured before and/or after receiving said concentrated population of adipose-derived regenerative cells. By some of these approaches, the adipose derived regenerative cells are CD14 or CD11b positive. The adipose-derived regenerative cells (e.g., a cell composition that comprises a concentrated population of adipose-derived regenerative cells that comprises stem cells) can be cultured cells but preferably, they are not cultured and are used after isolation (e.g., freshly isolated cells). That is, aspects of the invention concern the use of a therapeutically effective amount of a concentrated population of adipose derived regenerative cells to prepare a medicament for the treatment of pancreatitis, wherein said concentrated population of cells is to be administered to a patient in need thereof without culturing the cells before administering them to the patient and the adipose derived regenerative cells can be CD14 positive or CD11b positive.

### BRIEF DESCRIPTION OF THE DRAWINGS

FIG. 1 illustrates H&E stains of healthy pancreata from mice without pancreatitis.

FIG. 2 illustrates Representative H&E stains of pancreata in high power fields (400× magnification). Mice were injected with 1×10⁶ ADRCs (Panels A and C) or PBS (Panels B and D) after induction of pancreatitis and tissue excised after 48 hours (Panels A and B) or 72 hours (Panels C and D). Signs of pancreatitis are variable, but include necrosis, edema, and infiltration in each group. Despite variability between samples, note that the general pathology of ADRC-treated pancreata appear more healthy than those treated with PBS. Compare to controls in FIG. 1.

FIG. 3 ADRCs attenuate acinar cell necrosis and perivascular infiltration in mice with pancreatitis at 48 hours. Two independent and blinded investigators (J. R. and S. X) scored necrosis and perivascular cell infiltrate on identical, randomized, high powered fields. Three fields from each pancreas of ADRC-treated animals (n=35) and PBS-treated animals (n=39) were scored. Both investigators found significant differences between cell-treated and untreated mice for each parameter. Error bars represent standard deviation.

FIG. 4 ADRCs attenuate acinar cell necrosis and perivascular infiltration in mice with pancreatitis at 72 hours. Two independent and blinded investigators (J. R. and S. X) scored necrosis and perivascular cell infiltrate on identical, randomized, high powered fields. Three fields from each pancreas of ADRC-treated animals (n=31) and PBS-treated animals (n=27) were scored. Both investigators found significant differences between cell-treated and untreated mice for each parameter. Error bars represent standard deviation.

FIG. 5 Serum amylase concentrations following treatment with ADRCs or PBS. Serum was analyzed following terminal bleeding only in Experiment 1. No difference between the two therapies was detected in the second experiment (n=33 for ADRC-treatment, n=38 for control; p=0.14, error bars represent standard deviation).

FIG. 6 Serum lipase concentrations following treatment with ADRCs or PBS. Serum was analyzed following terminal bleeding only in Experiment 1. No difference between the two therapies was detected (n=16 for ADRC-treatment, n=18 for control; p=0.48, error bars represent standard deviation).

FIG. 7 Serum creatinine levels following treatment with ADRCs.

FIG. 8 Blood Urea Nitrogen levels following treatment with ADRCs.

FIG. 9 Serum creatinine levels following treatment with cryppreserved ADRCs.

FIG. 10 CD marker based subpopulations in fresh and thawed ADRCs.

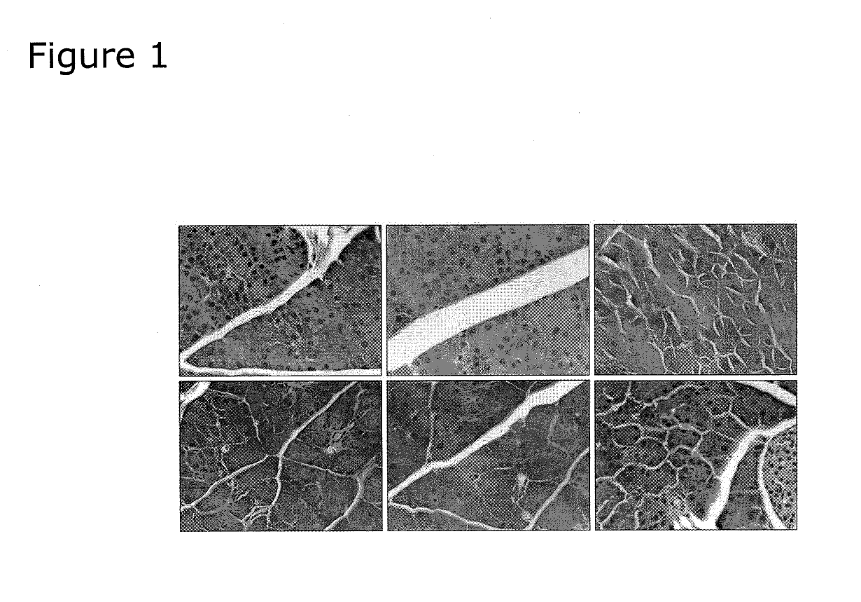

### DETAILED DESCRIPTION OF THE PREFERRED EMBODIMENT

Embodiments disclosed herein relate to the discovery that adipose tissue is a rich source of cells capable of modulating inflammation and thereby reducing the damage incurred in pancreatic acinar cells as well as the inflammatory infiltrate (perivascular infiltrate) due to pancreatitis. Embodiments disclosed herein also relate to the discovery that therapies using adipose derived cells mitigate, inhibit, or reduce macrophage infiltration, as well as, other markers for inflammation and down regulate inflammatory related gene expression (e.g., in acute kidney injury). Embodiments disclosed herein provide methods for administration of adipose tissue and/or components of adipose tissue to modulate inflammation (e.g., inhibit or reduce the presence of a marker associated with inflammation) and to treat or ameliorate, for example, pancreatitis, acute kidney injury, and conditions associated with these disease states. In some embodiments, adipose tissue and/or adipose-derived regenerative cells (ADRCs) are administered to a patient suffering from pancreatitis in an amount sufficient to reduce the level of a marker for inflammation (e.g., a cytokine such as, an interleukin, an interferon, a Chemokine (C-X-C motif) ligand 2 (CXCL2) or a growth factor such as, hepatocyte growth factor (HGF), Transforming Growth Factor alpha or beta (TGFα or β), Tumor Necrosis Factor alpha, or Vascular endothelial Growth Factor (VEGF))-. In further embodiments, the adipose tissue and adipose-derived regenerative cells are administered in combination. The implant can reduce the histopathological indications of pancreatits, such as damage incurred in pancreatic acinar cells and the inflammatory infiltrate due to pancreatitis.

In some contexts, the term “adipose tissue” refers to a tissue containing multiple cell types including adipocytes and vascular cells. Adipose tissue includes multiple regenerative cell types, including adult stem cells (ASCs) and endothelial progenitor and precursor cells. Accordingly, adipose tissue refers to fat, including the connective tissue that stores the fat.

In some contexts, the term “unit of adipose tissue” refers to a discrete or measurable amount of adipose tissue. A unit of adipose tissue may be measured by determining the weight and/or volume of the unit. In reference to the disclosure herein, a unit of adipose tissue may refer to the entire amount of adipose tissue removed from a subject, or an amount that is less than the entire amount of adipose tissue removed from a subject. Thus, a unit of adipose tissue may be combined with another unit of adipose tissue to form a unit of adipose tissue that has a weight or volume that is the sum of the individual units.

In some contexts, the term “portion” refers to an amount of a material that is less than a whole. A minor portion refers to an amount that is less than 50%, and a major portion refers to an amount greater than 50%. Thus, a unit of adipose tissue that is less than the entire amount of adipose tissue removed from a subject is a portion of the removed adipose tissue.

As used herein, “regenerative cells” refers to any heterogeneous or homologous cells obtained using the systems and methods of embodiments disclosed herein which cause or contribute to complete or partial regeneration, restoration, or substitution of structure or function of an organ, tissue, or physiologic unit or system to thereby provide a therapeutic, structural or cosmetic benefit. Examples of regenerative cells include: ASCs, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes.

In some contexts, the term “progenitor cell” refers to a cell that is unipotent, bipotent, or multipotent with the ability to differentiate into one or more cell types, which perform one or more specific functions and which have limited or no ability to self-renew. Some of the progenitor cells disclosed herein may be pluripotent.

In some contexts, the term “adipose tissue-derived cells” refers to cells extracted from adipose tissue that has been processed to separate the active cellular component from the mature adipocytes and connective tissue. Separation may be partial or full. That is, the “adipose tissue-derived cells” may or may not contain some adipocytes and connective tissue. This fraction is referred to herein as “adipose tissue-derived cells,” “adipose derived cells,” “adipose derived regenerative cells” or “ADC.” Typically, ADC refers to the pellet of cells obtained by washing and separating the cells from the adipose tissue. The pellet is typically obtained by centrifuging a suspension of cells so that the cells aggregate at the bottom of a centrifuge container.

In some contexts, “inflammation” indicates a process by which the body's white blood cells and chemicals are activated to protect the body from infection and foreign substances such as bacteria and viruses. In some other contexts, “inflammation” refers to the process by which the white blood cells and their inflammatory chemicals cause damage to the body's tissues. In some of these contexts, the body's immune system inappropriately triggers an inflammatory response despite the absence of injurious stimuli, e.g., in autoimmune diseases. For example arthritis is an autoimmue disease that has misdirected inflammation. In some contexts, inflammation affects internal organs, including but not limited to, inflammation of the heart, inflammation in the lungs, inflammation in the kidneys, inflammation of the large intestine, etc.

In some contexts, inflammation is a mechanism that contributes to acute kidney disease. Kidney disease is a leading cause of morbidity and mortality in hospitalized patients and represents an annual cost of at least $32 billion for the care for end stage renal disease alone, representing more than a quarter of annual Medicare expenditures. Currently, Acute Kidney Injury (AKI) is diagnosed in over 300,000 Americans annually and is defined by an abrupt and sustained impairment of renal function that can be initiated by various insults, including ischemia, bacterial infections and nephrotoxins. Renal ischemia is often a secondary result of procedures such as cardiopulmonary bypass, nephron sparing surgery and kidney transplantation and is the most common initiator of AKI.⁶⁻⁹ AKI is associated with prolonged hospitalization, marked increases in morbidity as well as early and late mortality.

Several mechanisms contribute to the pathogenesis of AKI following renal ischemia. These include inflammation. Despite advances in modern medical technology, no effective therapies for AKI beyond supportive treatment are currently available.

In some contexts, the term “pancreatitis” indicates an inflammatory disease which is a disease of pancreas whose major causes include excessive alcohol consumption and ductal obstruction (e.g. by gallstones) and whose presentation reflects a continuum of morphologic abnormalities that may include glandular inflammation of pancreas. In the acute stage, this ranges from mild disease (edematous pancreatitis) to the severe form (hemorrhagic or necrotizing pancreatitis). The former is characterized by exudation of neutrophils and interstitial edema with apparent preservation of parenchymal elements, the latter by coagulation necrosis of the gland and surrounding fatty tissue, resulting in loss of structural integrity, and, possibly, bleeding. Severe acute pancreatitis is usually a result of pancreatic glandular necrosis. The morbidity and mortality associated with acute pancreatitis are substantially higher when necrosis is infected (i.e., “infected acute pancreatitis”). Acute pancreatitis usually has a rapid onset manifested by upper abdominal pain, vomiting, fever, tachycardia, leukocytosis, and elevated serum levels of pancreatic enzymes. The disclosed method can be used to treat all of these forms of pancreatitis.

The major causes of acute pancreatitis are alcohol abuse and gallstones, which together account for approximately 75% of all cases. Other causes include drugs such as imuran, DDI and pentamidine, infections such as CMV, hypertriglyceridemia, hypercalcemia and hypotension. Pancreatitis can also have mechanical causes such as ductal obstructions which commonly occur in patients with carcinoma of the pancreas, post-operative and post endoscopic retrograde cholangiopancreatography (post-ERCP) as well as trauma-related causes.

Acute pancreatitis can be induced by alcohol ingestion, biliary tract disease (gallstones), postoperative state (after abdominal or nonabdominal operation), endoscopic retrograde cholangiopancreatography (ERCP), especially manometric studies of sphincter of Oddi, trauma (especially blunt abdominal type), or metabolic causes such as hypertriglyceridemia, apolipoprotein CII deficiency syndrome, hypercalcemia (e.g., hyperparathyroidism), renal failure drug-induced or as a result of renal transplantation, or acute fatty liver of pregnancy. The acute pancreatitis can be a hereditary pancreatitis or can be caused by infections such as mumps, viral hepatitis, other viral infections including coxsackievirus, echovirus, and cytomegalovirus, ascariasis, or infections with Mycoplasma, Campylobacter, Mycobacterium avium complex. Pancreatitis can also be induced by medicaments or drugs such as azathioprine, 6-mercaptopurine, sulfonamides, furosemide, thiazide diuretics, estrogens (oral contraceptives), tetracycline, pentamidine, valproic acid, dideoxyinosine, acetaminophen, nitrofurantoin, erythromycin, methyldopa, salicylates, metronidazole, nonsteroidal anti-inflammatory drugs, or angiotensin-converting enzyme (ACE) inhibitors. The method of the present invention can also be used to treat pancreatitis or to reduce or alleviate at least one adverse effect or symptom of a pancreatic condition, disease or disorder (e.g., any disorder characterized by abnormal, anomalous or insufficient pancreatic function, such as, acute pancreatitis, caused by ischemic-hypoperfusion state (after cardiac surgery), atherosclerotic emboli, systemic lupus erythematosus, necrotizing angiitis, trombotic thrombocytopenic purpura, penetrating peptic ulcer, obstruction of the ampulla of Vater, regional enteritis, duodenal diverticulum, or pancreas divisum.*-

In some contexts, the term “expansion,” is used to encompass repair, regeneration, proliferation, differentiation, migration, survival, or any growth parameter of any pancreatic structure, including acinar cells and any structures composed in whole or in part of pancreatic cells. Cells that enhance expansion of the pancreatic system are cells that enhance expansion of the pancreatic system by any mechanism, either direct or indirect. “Modulation of expansion” is meant to encompass an influencing expansion in either a stimulatory or inhibitory manner, as is necessary for treating a disorder characterized by anomalous, abnormal, undesirable, or insufficient pancreatic function. It is understood that the various functions or components of the pancreatic system can become more or less active, and therefore can require different levels of modulation, at different times, even within the same patient. These requirements are affected, e.g., by disease type, disease stage, patient variation due to age, gender, health status, genetic factors, environmental factors, drugs and combinations of drugs administered currently or formerly to the patient, etc.

In some contexts, the term “treating” includes reducing or alleviating at least one adverse effect or symptom of a pancreatic condition, disease or disorder, e.g., any disorder characterized by abnormal, anomalous or insufficient pancreatic function (e.g, acute pancreatitis). For example, adverse effects or symptoms of pancreatitis or markers of inflammation, in general, are well-known in the art and include, but are not limited to, abdominal pain, swollen and tender abdomen, nausea, vomiting, fever, rapid pulse, dehydration, and low blood pressure. Markers of inflammation also include macrophage infiltration at a site, the amount or level of a cytokine such as, an interleukin (e.g., IL-3, IL-6, IL-8, or IL-12), an interferon (e.g., interferon gamma), a Chemokine (C-X-C motif) ligand 2 (CXCL2), or a growth factor such as, hepatocyte growth factor (HGF), Transforming Growth Factors (e.g., TGF alpha or TGF beta), Tumor Necrosis Factor alpha, or VEGF.

In some contexts, the terms “administering,” “introducing” and “transplanting” are used interchangeably and refer to the placement of a cell population as described herein into a subject by a method or route, which results in localization of a cell population, as described herein at a desired site. The cell population, as described herein, can be administered by any appropriate route which results in delivery to a desired location in the subject where at least a portion of the cells remain viable. The period of viability of the cells after administration to a subject can be as short as a few hours, e.g., twenty-four hours, to a few days, to as long as several years.

In some contexts, the term “subject” includes warm-blooded animals, preferably mammals, including humans. In a preferred embodiment, the subject is a primate. In a more preferred embodiment, the subject is a human.

In some contexts, the term “immunosuppressive drug or agent” is intended to include pharmaceutical agents, which inhibit or interfere with normal immune function. Examples of immunosuppressive agents suitable with the methods disclosed herein include agents that inhibit T-cell/B-cell co-stimulation pathways, such as agents that interfere with the coupling of T-cells and B-cells via the CTLA4 and B7 pathways, as disclosed in U.S. Pub. No. 2002/0182211. A preferred immunosuppressive agent is cyclosporin A. Other examples include myophenylate mofetil, rapamicin, and anti-thymocyte globulin. In one embodiment, the immunosuppressive drug is administered with at least one other therapeutic agent. The immunosuppressive drug can be administered in a formulation, which is compatible with the route of administration and is administered to a subject at a dosage sufficient to achieve the desired therapeutic effect. In another embodiment, the immunosuppressive drug is administered transiently for a sufficient time to induce tolerance to a cell population described herein.

In some contexts, the phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

In some contexts, the term “unit dose” is used to refer to a discrete amount of a therapeutic composition dispersed in a suitable carrier. Those of ordinary skill in the art will readily optimize effective dosages and administration regimens as determined, e.g., by good medical practice and the characteristics of the individual patient. Further refinement of dosages can be made by those of ordinary skill in the art based, e.g., on data observed in animals or human clinical trials. The section below describes several approaches to obtain, refine, enrich, concentrate, isolate, or purify ADRCs.

### Methods of Making an Adipose-Derived Cell Population Comprising ADRCs

In some embodiments, adipose tissue is processed to obtain a refined, enriched, concentrated, isolated, or purified population of ADRCs using a cell processing unit, gradient sedimentation, filtration, or a combination of any one or more of these approaches. In general, adipose tissue is first removed from a subject (e.g., a mammal, a domestic animal, a rodent, a horse, a dog, cat, or human) then it is processed to obtain a cell population comprising ADRCs. For allogeneic transplantation, an appropriate donor can be selected using methods known in the art, for example, methods used for selection of bone marrow donors. The volume of adipose tissue collected from the patient can vary from about 1 cc to about 2000 cc and in some embodiments up to about 3000 cc. The volume of tissue removed will vary from patient to patient and will depend on a number of factors including but not limited to: age, body habitus, coagulation profile, hemodynamic stability, severity of insufficiency or injury, co-morbidities, and physician preference.

The adipose tissue can be obtained by any method known to a person of ordinary skill in the art. For example, the adipose tissue may be removed from a subject by suction-assisted lipoplasty, ultrasound-assisted lipoplasty, or excisional lipectomy. In addition, the procedures may include a combination of such procedures, such as a combination of excisional lipectomy and suction-assisted lipoplasty. If the tissue or some fraction thereof is intended for re-implantation into a subject, the adipose tissue should be collected in a manner that preserves the viability of the cellular component and that minimizes the likelihood of contamination of the tissue with potentially infectious organisms, such as bacteria and/or viruses. Thus, the tissue extraction should be performed in a sterile or aseptic manner to minimize contamination. Suction-assisted lipoplasty may be desired to remove the adipose tissue from a patient as it provides a minimally invasive method of collecting tissue with minimal potential for stem cell damage that may be associated with other techniques, such as ultrasound-assisted lipoplasty.

Accordingly, adipose tissue provides a rich source of a population of cells that is easily enriched for ADRCs. Collection of adipose tissue is also more patient-friendly and is associated with lower morbidity than collection of a similar volume of, for example, skin or a much larger volume of tonsil.

For suction-assisted lipoplastic procedures, adipose tissue is collected by insertion of a cannula into or near an adipose tissue depot present in the patient followed by aspiration of the adipose into a suction device. In some embodiments, a small cannula may be coupled to a syringe, and the adipose tissue may be aspirated using manual force. Using a syringe or other similar device may be desirable to harvest relatively moderate amounts of adipose tissue (e.g., from 0.1 ml to several hundred milliliters of adipose tissue). Procedures employing these relatively small devices require only local anesthesia. Larger volumes of adipose tissue (e.g., greater than several hundred milliliters) may require general anesthesia at the discretion of the donor and the person performing the collection procedure. When larger volumes of adipose tissue are to be removed, relatively larger cannulas and automated suction devices may be employed.

Excisional lipectomy procedures include, and are not limited to, procedures in which adipose tissue-containing tissues (e.g., skin) is removed as an incidental part of the procedure; that is, where the primary purpose of the surgery is the removal of tissue (e.g., skin in bariatric or cosmetic surgery) and in which adipose tissue is removed along with the tissue of primary interest. Subcutaneous adipose tissue may also be extracted by excisional lipectomy in which the adipose tissue is excised from the subcutaneous space without concomitant removal of skin.

The amount of tissue collected can depend on a number of variables including, but not limited to, the body mass index of the donor, the availability of accessible adipose tissue harvest sites, concomitant and pre-existing medications and conditions (such as anticoagulant therapy), and the clinical purpose for which the tissue is being collected. Experience with transplant of hematopoietic stem cells (bone marrow or umbilical cord blood-derived stem cells used to regenerate the recipient's blood cell-forming capacity) shows that engraftment is cell dose-dependent with threshold effects (Smith, et al., 1995; Barker, et al., 2001, both incorporated herein by reference in their entirety). Thus, it is possible that the general principle that “more is better” will be applied within the limits set by other variables and that where feasible the harvest will collect as much tissue as possible.

The adipose tissue that is removed from a patient is then collected into a device (e.g., cell processing unit, centrifuge, or filtration unit) for further processing so as to remove collagen, adipocytes, blood, and saline, thereby obtaining a cell population comprising ADRCs. Preferably the population of adipose derived cells containing ADRCs is free from contaminating collagen, adipocytes, blood, and saline. The major contaminating cells in adipose tissue (adipocytes) have low density and are easily removed by flotation.

Adipose tissue processing to obtain a refined, concentrated, and isolated population of ADRCs and modifications thereto are preferably performed using methods described, for example, in U.S. application Ser. No. 10/316,127 (U.S. Pat. App. Pub. No. 2003/0161816), entitled SYSTEMS AND METHODS FOR TREATING PATIENTS WITH PROCESSED LIPOASPIRATE CELLS, filed Dec. 9, 2002, and U.S. application Ser. No. 10/877,822 (U.S. Pat. App. Pub. No. 2005/0084961), entitled SYSTEMS AND METHODS FOR SEPARATING AND CONCENTRATING REGENERATIVE CELLS FROM TISSUE, filed Jun. 25, 2004; U.S. application Ser. No. 10/242,094, entitled PRESERVATION OF NON EMBRYONIC CELLS FROM NON HEMATOPOIETIC TISSUES, filed Sep. 12, 2002, which claims the benefit of U.S. application Ser. No. 60/322,070 filed Sep. 14, 2001; U.S. application Ser. No. 10/884,638, entitled SYSTEMS AND METHODS FOR ISOLATING AND USING CLINICALLY SAFE ADIPOSE DERIVED REGENERATIVE CELLS, filed on Jul. 2, 2004; all of which are hereby expressly incorporated by reference in their entireties. The applications above disclose the processing of adipose-derived cells in a system that is configured to maintain a closed, sterile fluid/tissue pathway. This can be achieved by use of a pre-assembled, linked set of closed, sterile containers and tubing allowing for transfer of tissue and fluid elements within a closed pathway. This processing set can be linked to a series of processing reagents (e.g., saline, enzymes, etc.) inserted into a device, which can control the addition of reagents, temperature, and timing of processing thus relieving operators of the need to manually manage the process. In a preferred embodiment, the entire procedure from tissue extraction through processing and placement into the recipient is performed in the same facility, indeed, even within the same room, of the patient undergoing the procedure.

For many applications, preparation of the active cell population requires depletion of the mature fat-laden adipocyte component of adipose tissue. This can be achieved by a series of washing and disaggregation steps in which the tissue is first rinsed to reduce the presence of free lipids (released from ruptured adipocytes) and peripheral blood elements (released from blood vessels severed during tissue harvest), and then disaggregated to free intact adipocytes and other cell populations from the connective tissue matrix. In some embodiments, ADRCs are provided with BECs, BEC progenitors (EPCs), and adipose tissue-derived stem cells, adipose tissue-derived stromal cells, and other cellular elements.

Rinsing is an optional but preferred step, wherein the tissue is mixed with a solution to wash away free lipid and single cell components, such as those components in blood, leaving behind intact adipose tissue fragments. In one embodiment, the adipose tissue that is removed from the patient is mixed with isotonic saline or other physiologic solution(s), e.g., Plasmalyte® of Baxter Inc. or Normoso® of Abbott Labs. Intact adipose tissue fragments can be separated from the free lipid and cells by any means known to persons of ordinary skill in the art including, but not limited to, filtration, decantation, sedimentation, or centrifugation. In some embodiments, the adipose tissue is separated from non-adipose tissue by employing a filter disposed within a tissue collection container, as discussed herein. In other embodiments, the adipose tissue is separated from non-adipose tissue using a tissue collection container that utilizes decantation, sedimentation, and/or centrifugation techniques to separate the materials.

The intact tissue fragments are then disaggregated using any conventional techniques or methods, including mechanical force (mincing or shear forces), ultrasonic or other physical energy, lasers, microwaves, enzymatic digestion with single or combinatorial proteolytic enzymes, such as collagenase, trypsin, lipase, liberase H1, nucleases, or members of the Blendzyme family as disclosed in U.S. Pat. No. 5,952,215, “Enzyme composition for tissue dissociation,” expressly incorporated herein by reference in its entirety, and pepsin, or a combination of mechanical and enzymatic methods. For example, the cellular component of the intact tissue fragments may be disaggregated by methods using collagenase-mediated dissociation of adipose tissue, similar to the methods for collecting microvascular endothelial cells in adipose tissue, as disclosed in U. S. Pat. No. 5,372,945, expressly incorporated herein by reference in its entirety. Additional methods using collagenase that may be used are disclosed in, e.g., U.S. Pat. Nos. 5,830,741, “Composition for tissue dissociation containing collagenase I and II from clostridium histolyticum and a neutral protease” and by Williams, et al., 1995, “Collagenase lot selection and purification for adipose tissue digestion,” Cell Transplant 4(3):281-9, both expressly incorporated herein by reference in their entirety. Similarly, a neutral protease may be used instead of collagenase, as disclosed in Twentyman, et al. (Twentyman, et al., 1980, “Use of bacterial neutral protease for disaggregation of mouse tumours and multicellular tumor spheroids,” Cancer Lett. 9(3):225-8, expressly incorporated herein by reference in its entirety). Furthermore, the methods described herein may employ a combination of enzymes, such as a combination of collagenase and trypsin or a combination of an enzyme, such as trypsin, and mechanical dissociation.

Adipose tissue-derived cells may then be obtained from the disaggregated tissue fragments by reducing the number of mature adipocytes. A suspension of the disaggregated adipose tissue and the liquid in which the adipose tissue was disaggregated is then passed to another container, such as a cell collection container. The suspension may flow through one or more conduits to the cell collection container by using a pump, such as a peristaltic pump, that withdraws the suspension from the tissue collection container and urges it to the cell collection container. Other embodiments may employ the use of gravity or a vacuum while maintaining a closed system. Separation of the cells in the suspension may be achieved by buoyant density sedimentation, centrifugation, elutriation, filtration, differential adherence to and elution from solid phase moieties, antibody-mediated selection, differences in electrical charge, immunomagnetic beads, fluorescence activated cell sorting (FACS), or other means. Examples of these various techniques and devices for performing the techniques may be found in U.S. Pat. Nos. 6,277,060; 6,221,315; 6,043,066; 6,451,207; 5,641,622; and 6,251,295, all incorporated herein by reference in their entirety. Many of these devices can be incorporated within the cell processing unit, while maintaining a closed system.

In some embodiments, the cells in the suspension are separated from the acellular component of the suspension using a spinning membrane filter. In other embodiments, the cells in the suspension are separated from the acellular component using a centrifuge. In one such exemplary embodiment, the cell collection container may be a flexible bag that is structured to be placed in a centrifuge (e.g., manually or by robotics). In other embodiments, a flexible bag is not used. After centrifugation, the cellular component containing ADRCs forms a pellet, which may then be resuspended with a buffered solution so that the cells can be passed through one or more conduits to a mixing container, as discussed herein. The resuspension fluids may be provided by any suitable means. For example, a buffer may be injected into a port on the cell collection container, or the cell collection container may include a reserve of buffer that can be mixed with the pellet of cells by rupturing the reserve. When a spinning membrane filter is used, resuspension is optional since the cells remain in a volume of liquid after the separation procedure.

Although some embodiments described herein are directed to methods of fully disaggregating the adipose tissue to separate the active cells from the mature adipocytes and connective tissue, additional embodiments are directed to methods in which the adipose tissue is only partially disaggregated. For example, partial disaggregation may be performed with one or more enzymes, which are removed from at least a part of the adipose tissue early relative to an amount of time that the enzyme would otherwise be left thereon to fully disaggregate the tissue. Such a process may require less processing time and would generate fragments of tissue components within which multiple ADRCs remain in partial or full contact.

In some embodiments, the tissue is washed with sterile buffered isotonic saline and incubated with collagenase at a collagenase concentration, a temperature, and for a period of time sufficient to provide adequate disaggregation. In a preferred embodiment, the collagenase enzyme used will be approved for human use by the relevant authority (e.g., the U.S. Food and Drug Administration). Suitable collagenase preparations include recombinant and non-recombinant collagenase. Non-recombinant collagenase may be obtained from F. Hoffmann-La Roche Ltd., Indianapolis, IN and/or Advance Biofactures Corp., Lynbrook, N.Y. Recombinant collagenase may also be obtained as disclosed in U.S. Pat. No. 6,475,764.

In one embodiment, solutions contain collagenase at concentrations of about 10 μg/ml to about 50 μg/ml (e.g., 10 μg/ml, 20 μg/ml, 30 μg/ml, 40 μg/ml, or 50 μg/ml) and are incubated at from about 30° C. to about 38° C. for from about 20 minutes to about 60 minutes. These parameters will vary according to the source of the collagenase enzyme, optimized by empirical studies, in order to confirm that the system is effective at extracting the desired cell populations in an appropriate time frame. A particular preferred concentration, time and temperature is 20 μg/ml collagenase (mixed with the neutral protease dispase; Blendzyme 1, Roche) and incubated for 45 minutes at about 37° C. An alternative preferred embodiment applies 0.5 units/mL collagenase (mixed with the neutral protease thermolysin; Blendzyme 3). In a particularly preferred embodiment the collagenase enzyme used is material approved for human use by the relevant authority (e.g., the U.S. Food and Drug Administration). The collagenase used should be free of micro-organisms and contaminants, such as endotoxin.

Following disaggregation the active cell population can be washed/rinsed to remove additives and/or by-products of the disaggregation process (e.g., collagenase and newly-released free lipid). The active cell population can then be concentrated by centrifugation or other methods known to persons of ordinary skill in the art, as discussed above. These post-processing wash/concentration steps may be applied separately or simultaneously. In one embodiment, the cells are concentrated and the collagenase removed by passing the cell population through a continuous flow spinning membrane system or the like, such as, for example, the system disclosed in U.S. Pat. Nos. 5,034,135 and 5,234,608, all incorporated herein by reference in their entirety.

In addition to the foregoing, there are many known post-wash methods that may be applied for further purifying the adipose-derived cell population that comprises ADRCs. These include both positive selection (selecting the target cells), negative selection (selective removal of unwanted cells), or combinations thereof. In addition to separation by flow cytometry as described herein and in the literature, cells can be separated based on a number of different parameters, including, but not limited to, charge or size (e.g., by dielectrophoresis or various centrifugation methods, etc.).

Many other conformations of the staged mechanisms used for cell processing will be apparent to one skilled in the art. For example, mixing of tissue and saline during washing and disaggregation can occur by agitation or by fluid recirculation. Cell washing may be mediated by a continuous flow mechanism such as the spinning membrane approach, differential adherence, differential centrifugation (including, but not limited to differential sedimentation, velocity, or gradient separation), or by a combination of means. Similarly, additional components allow further manipulation of cells, including addition of growth factors or other biological response modifiers, and mixing of cells with natural or synthetic components intended for implant with the cells into the recipient.

Post-processing manipulation may also include cell culture or further cell purification (Kriehuber, et al., 2001; Garrafa, et al., 2006). In some embodiments, once the adipose-derived cell population that comprises ADRCs is obtained, it is further refined, concentrated, enriched, isolated, or purified using a cell sorting device and/or gradient sedimentation. Mechanisms for performing these functions may be integrated within the described devices or may be incorporated in separate devices. In many embodiments, however, a therapeutically effective amount of a concentrated population of adipose derived regenerative cells is used to prepare a medicament for the reduction of inflammation (e.g., pancreatitis), wherein said concentrated population of cells is to be administered to a patient in need thereof without culturing the cells before administering them to the patient. That is, some embodiments concern methods to reduce inflammation and/or to treat, inhibit, or ameliorate pancreatitis or acute kidney injury or a condition associated therewith, wherein a therapeutically effective amount of a concentrated population of adipose derived regenerative cells is administered to a patient in need thereof without culturing the cells before administering them to the patient.

In a preferred embodiment of the invention, the tissue removal system and processing set would be present in the vicinity of the patient receiving the treatment, such as the operating room or out-patient procedure room (effectively at the patient's bedside). This allows rapid, efficient tissue harvest and processing, and decreases the opportunity for specimen handling/labeling error, thereby allowing for performance of the entire process in the course of a single surgical procedure.

As described in U.S. application Ser. No. 10/884,638, entitled SYSTEMS AND METHODS FOR ISOLATING AND USING CLINICALLY SAFE ADIPOSE DERIVED REGENERATIVE CELLS, filed on Jul. 2, 2004, one or more additives may be added to the cells during and/or after processing. Some examples of additives include agents that optimize washing and disaggregation, additives that enhance the viability of the active cell population during processing, anti-microbial agents (e.g., antibiotics), additives that lyse adipocytes and/or red blood cells, or additives that enrich for cell populations of interest (by differential adherence to solid phase moieties or to otherwise promote the substantial reduction or enrichment of cell populations).

The ADRCs obtained as described herein can be cultured according to approaches known in the art, and the cultured cells can be used in several of the embodied methods. For example, ADRCs can be cultured on collagen-coated dishes or 3D collagen gel cultures in endothelial cell basal medium in the presence of low or high fetal bovine serum or similar product, as described in Ng, et al., November 2004, “Interstitial flow differentially stimulates blood and lymphatic endothelial cell morphogenesis in vitro,” Microvasc Res. 68(3):258-64, incorporated herein by reference. Alternatively, ADRCs can be cultured on other extracellular matrix protein-coated dishes. Examples of extracellular matrix proteins that may be used include, but are not limited to, fibronectin, laminin, vitronectin, and collagen IV. Gelatin or any other compound or support, which similarly promotes adhesion of endothelial cells into culture vessels may be used to culture ADRCs, as well.

Examples of basal culture medium that can be used to culture ADRCs in vitro include, but are not limited to, EGM, RPMI, M199, MCDB131, DMEM, EMEM, McCoy's 5A, Iscove's medium, modified Iscove's medium or any other medium known in the art to support the growth of blood endothelial cells. Examples of supplemental factors or compounds that can be added to the basal culture medium that could be used to culture ADRCs include, but are not limited to, ascorbic acid, heparin, endothelial cell growth factor, endothelial growth supplement, glutamine, HEPES, Nu serum, fetal bovine serum, human serum, equine serum, plasma-derived horse serum, iron-supplemented calf serum, penicillin, streptomycin, amphotericin B, basic and acidic fibroblast growth factors, insulin-growth factor, astrocyte conditioned medium, fibroblast or fibroblast-like cell conditioned medium, sodium hydrogencarbonate, epidermal growth factor, bovine pituitary extract, magnesium sulphate, isobutylmethylxanthine, hydrocortisone, dexamethasone, dibutyril cyclic AMP, insulin, transferrin, sodium selenite, oestradiol, progesterone, growth hormone, angiogenin, angiopoietin-1, Del-1, follistatin, granulocyte colony-stimulating factor (G-CSF), erythropoietin, hepatocyte growth factor (HGF)/scatter factor (SF), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular endothelial growth factor (VEGF)/vascular permeability factor (VPF), interleukin-3 (IL-3), interleukin 7 (IL-7), interleukin-8 (IL-8), ephrins, matrix metalloproteinases (such as MMP2 and MMP9), or any other compound known in the art to promote survival, proliferation or differentiation of endothelial cells.

Further processing of the cells may also include: cell expansion (of one or more regenerative cell types) and cell maintenance (including cell sheet rinsing and media changing); sub-culturing; cell seeding; transient transfection (including seeding of transfected cells from bulk supply); harvesting (including enzymatic, non-enzymatic harvesting and harvesting by mechanical scraping); measuring cell viability; cell plating (e.g., on microtiter plates, including picking cells from individual wells for expansion, expansion of cells into fresh wells); high throughput screening; cell therapy applications; gene therapy applications; tissue engineering applications; therapeutic protein applications; viral vaccine applications; harvest of regenerative cells or supernatant for banking or screening, measurement of cell growth, lysis, inoculation, infection or induction; generation of cell lines (including hybridoma cells); culture of cells for permeability studies; cells for RNAi and viral resistance studies; cells for knock-out and transgenic animal studies; affinity purification studies; structural biology applications; assay development and protein engineering applications.

In general, a system useful for isolating a cell population comprising ADRCs comprises a) a tissue collection container including i) a tissue collecting inlet port structured to receive adipose tissue removed from a subject, and ii) a filter disposed within the tissue collection container, which is configured to retain a cell population ADRCs from said subject and to pass adipocytes, blood, and saline; b) a mixing container or cell processing chamber coupled to the tissue collection container by a conduit such that a closed pathway is maintained, wherein said mixing container receives said cell population comprising ADRCs and said mixing container comprises an additive port for introducing at least one additive to said cell population comprising ADRCs; and an outlet port configured to allow removal of said cell population comprising ADRCs from the mixing container or cell processing chamber for administration to a patient. In some embodiments, said mixing container or cell processing container further comprises a cell concentration device such as a spinning membrane filter and/or a centrifuge. Aspects of the invention also include a cell sorter, which is attached to said mixing chamber or cell processing chamber by a conduit and is configured to receive cells from said mixing chamber or cell processing chamber, while maintaining a closed pathway. Aspects of the embodiments above may also include a centrifuge attached to said mixing chamber or cell processing chamber by a conduit and configured to receive said cell population comprising ADRCs, while maintaining a closed pathway, wherein said centrifuge comprises a gradient suitable for further separation and purification of said ADRCs (e.g., ficoll-hypaque). Said centrifuge containing said gradient, which is configured to receive said cell population comprising ADRCs may also be contained within said mixing container or cell processing chamber.

### Measuring ADRCs and ADRC Subsets in an Isolated Cell Population

A measurement, analysis, or characterization of said ADRCs to determine the presence of these cells in a cell population can be undertaken within the closed system of a cell processing unit or outside of the closed system of a cell processing unit using any number of protein and/or RNA detection assays available in the art. Additionally, the measurement, analysis, or characterization of said ADRCs can be part of or can accompany the isolation procedure (e.g., cell sorting using an antibody specific for ADRCs or gradient separation using a media selective for ADRCs).

In some embodiments the measurement or characterization of the isolated cell population is conducted by detecting the presence or absence of a protein marker that is unique to ADRCs or is otherwise considered to confirm the presence of ADRCs by those of skill in the art. In addition to conventional Western blots using antibody probes specific for said proteins or markers, immunoselection techniques that exploit on cell surface marker expression can be performed using a number of methods known in the art and described in the literature. Such approaches can be performed using an antibody that is linked directly or indirectly to a solid substrate (e.g., magnetic beads) in conjunction with a manual, automated, or semi-automated device as described by Watts, et al., for separation of CD34-positive cells (Watts, et al., 2002, Variable product purity and functional capacity after CD34 selection: a direct comparison of the C1iniMACS (v2.1) and Isolex 300i (v2.5) clinical scale devices,” Br J Haematol. 2002 July; 118(1):117-23), by panning, use of a Fluorescence Activated Cell Sorter (FACS), or other means.

Separation, measurement, and characterization can also be achieved by positive selection using antibodies that recognize cell surface markers or marker combinations that are expressed by ADRCs, but not by one or more of the other cell sub-populations present within the cell population. Separation, measurement, and characterization can also be achieved by negative selection, in which non-ADRCs are removed from the isolated cell population using antibodies or antibody combinations that do not exhibit appreciable binding to ADRCs. Markers that are specifically expressed by ADRCs have been described. Examples of antibodies that could be used in negative selection include, but are not limited to, markers expressed by endothelial cells. There are many other antibodies well known in the art that could be applied to negative selection. The relative specificity of markers for ADRCs can also be exploited in a purification and/or characterization or measurement strategy. For example, a fluorescently-labeled ligand can be used in FACS-based sorting of cells, or an ligand conjugated directly or indirectly to a solid substrate can be used to separate in a manner analogous to the immunoselection approaches described above.

Measurement and characterization of the adipose-derived cell population to determine the presence or absence of ADRCs can also involve analysis of one or more RNAs that encode a protein that is unique to or otherwise considered by those of skill in the art to be a marker that indicates the presence or absence of a ADRCs. In some embodiments, for example, the isolated cell population or a portion thereof is analyzed for the presence or absence of an RNA that encodes one or more of, e.g., CD45, CD11b, CD14, CD68, CD90, CD73, CD31 and/or CD34. The detection of said RNAs can be accomplished by any techniques available to one of skill in the art, including but not limited to, Northern hybridization, PCR-based methodologies, transcription run-off assays, gene arrays, and gene chips.

### Compositions Comprising ADRCs and ADRC Subsets

In accordance with the aforementioned approaches, raw adipose tissue is processed to substantially remove mature adipocytes and connective tissue thereby obtaining a heterogeneous plurality of adipose tissue-derived cells comprising ADRCs suitable for placement within the body of a subject. The extracted ADRCs may be provided in a neat composition comprising these cells substantially free from mature adipocytes and connective tissue or in combination with an inactive ingredient (e.g., a carrier) or a second active ingredient (e.g., adipose-derived stem cell and/or adipose-derived endothelial cell). The cells may be placed into the recipient alone or in combination (e.g., in a single composition or co-administered) with biological materials, such as cells, tissue, tissue fragments, or stimulators of cell growth and/or differentiation, supports, prosthetics, or medical devices. The composition may include additional components, such as cell differentiation factors, growth promoters, immunosuppressive agents, or medical devices, as discussed herein, for example. In some embodiments, the cells, with any of the above mentioned additives, are placed into the person from whom they were obtained (e.g., autologous transfer) in the context of a single operative procedure with the intention of providing a therapeutic benefit to the recipient.

Accordingly, aspects of the invention include compositions that comprise, consist, or consist essentially of a refined, enriched, concentrated, isolated, or purified adipose-derived cell population comprising ADRCs and mixtures of these cells with a biological material, additive, support, prosthetic, or medical device, including but not limited to, unprocessed adipose tissue, collagen matrix or support, cell differentiation factors, growth promoters, immunosuppressive agents, processed adipose tissue containing adipose-derived stem cells and/or progenitor cells, and cell populations already containing an enriched amount of ADRCs. In some embodiments, the aforementioned compositions comprise an amount or concentration of refined, isolated, or purified ADRCs that is greater than or equal to 0.5%-1%, 1-2%, 2%-4%, 4%-6%, 6%-8%, 8%-10%, 10%-20%, 20%-30%, 30%-40%, 40%-50%, 50%-60%, 60%-70%, 70%-80%, 80%-90%, or 90%-100% ADRCs, as compared to the total adipose-tissue cell population. In some embodiments, the ADRCs express an amount of, e.g., CD45, CD11b, CD14, CD68, CD90, CD73, CD31 and/or CD34.

In some embodiments, the adipose-derived cell population that comprises ADRCs described herein is formulated in compositions that include at least one pharmaceutically acceptable diluent, adjuvant, or carrier substance, using any available pharmaceutical chemistry techniques. Generally, this entails preparing compositions that are essentially free of impurities that could be harmful to humans or animals.

Appropriate salts and buffers can be employed to stabilize and to facilitate uptake of the adipose-derived cell population that comprises ADRCs. Compositions contemplated herein can comprise an effective amount of the ADRCs in a pharmaceutically acceptable carrier or aqueous medium.

Administration of the compositions described herein can be via any common route so long as the target tissue is available via that route. Compositions administered according to the methods described herein may be introduced into the subject by, e.g., by intravenous, intradermal, intramuscular, intramammary, intraperitoneal, intrathecal, retrobulbar, intrapulmonary (e.g., term release); by oral, sublingual, nasal, anal, vaginal, or transdermal delivery, or by surgical implantation at a particular site. The introduction may consist of a single dose or a plurality of doses over a period of time. Vehicles for cell therapy agents are known in the art and have been described in the literature. See, for example Remington's Pharmaceutical Sciences, 18th Ed. (1990, Mack Publ. Co, Easton Pa. 18042) pp 1435-1712, incorporated herein by reference. Sterile solutions are prepared by incorporating the adipose-derived cell population that comprises ADRCs in the required amount in the appropriate buffer with or without various of the other components described herein.

Combination therapy with any two or more agents described herein also is contemplated as an aspect of the invention. Similarly, every combination of agents described herein, packaged together as a new kit, or formulated together as a single composition, is considered an aspect of the invention. Compositions for use according to aspects of the invention preferably include the adipose-derived cell population that comprises ADRCs formulated with a pharmaceutically acceptable carrier. The cells can also be applied with additives to enhance, control, or otherwise direct the intended therapeutic effect. For example, in some embodiments, the adipose-derived cell population that comprises ADRCs can be further purified by use of antibody-mediated positive and/or negative cell selection to enrich the cell population to increase efficacy, reduce morbidity, or to facilitate ease of the procedure. Similarly, cells can be applied with a biocompatible matrix, which facilitates in vivo tissue engineering by supporting and/or directing the fate of the implanted cells. In the same way, cells can be administered following genetic manipulation such that they express gene products that are believed to or are intended to promote the therapeutic response provided by the cells.

The adipose-derived cell population that comprises ADRCs can be applied alone or in combination with other cells, tissue, tissue fragments, growth factors, biologically active or inert compounds, resorbable plastic scaffolds, or other additive intended to enhance the delivery, efficacy, tolerability, or function of the population. The adipose-derived cell population that comprises ADRCs can also be modified by insertion of DNA or by placement in cell culture in such a way as to change, enhance, or supplement the function of the cells for derivation of a structural or therapeutic purpose.

In more embodiments, the adipose-derived cell population that comprises ADRCs are combined with a gene encoding a pro-drug converting enzyme which allows cells to activate pro-drugs within the site of engraftment, that is, within a tumor. Addition of the gene (or combination of genes) can be by any technology known in the art including but not limited to adenoviral transduction, “gene guns,” liposome-mediated transduction, and retrovirus or lentivirus-mediated transduction, plasmid, or adeno-associated virus. Cells can be implanted along with a carrier material bearing gene delivery vehicle capable of releasing and/or presenting genes to the cells over time such that transduction can continue or be initiated in situ. Particularly when the cells and/or tissue containing the cells are administered to a patient other than the patient from whom the cells and/or tissue were obtained, one or more immunosuppressive agents can be administered to the patient receiving the cells and/or tissue to reduce, and preferably prevent, rejection of the transplant.

Still more embodiments concern the ex vivo transfection of an adipose-derived cell population that comprises ADRCs and subsequent transfer of these transfected cells to subjects. It is contemplated that such embodiments can be an effective approach to upregulate in vivo levels of the transferred gene and for providing relief from a disease or disorder resulting from under-expression of the gene(s) or otherwise responsive to upregulation of the gene (see e.g., Gelse, et al., 2003, “Articular cartilage repair by gene therapy using growth factor-producing mesenchymal cells,” Arthritis Rheum. 48:430-41; Huard, et al, 2002, “Muscle-derived cell-mediated ex vivo gene therapy for urological dysfunction,” Gene Ther. 9:1617-26; Kim, et al., 2002, “Ex vivo gene delivery of IL-1Ra and soluble TNF receptor confers a distal synergistic therapeutic effect in antigen-induced arthritis,” Mol. Ther. 6:591-600, all incorporated herein by reference). Delivery of an adipose-derived cell population that comprises ADRCs to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, 1998, “Human Gene Therapy,” Nature Suppl. to vol. 392 (6679):25-20, incorporated by reference herein. Gene therapy technologies are also reviewed by Friedmann, 1989, “Progress toward human gene therapy,” Science 244(4910):1275-1281, Verma (1990), “Gene therapy.” Scientific American 263(5): 68-84, and Miller (1992), “Human gene therapy comes of age,” Nature, 357:455-460, all incorporated by reference herein. An adipose-derived cell population that comprises ADRCs can be cultured ex vivo in the presence of an additive (e.g., a compound that induces differentiation or pancreatic cell formation) in order to proliferate or to produce a desired effect on or activity in such cells. Treated cells can then be introduced to a subject.

In some embodiments, the ex vivo gene therapy is conducted locally, e.g., to the site of pancreatitis. For example, by using catheter-mediated transfer an adipose-derived cell population that comprises ADRCs can be transferred into a mammalian subject. Materials and methods for local delivery are reviewed, e.g., in Lincoff, et al. (1994), “Local drug delivery for the prevention of restenosis. Fact, fancy, and future,” Circulation, 90: 2070-2084, hereby expressly incorporated by reference. For example, adipose-derived cell population that comprises ADRCs can be provided to a subject by an infusion-perfusion balloon catheter (preferably a microporous balloon catheter), such as those that have been described in the literature for intracoronary drug infusions. See, e.g., U.S. Pat. No. 5,713,860 (Intravascular Catheter with Infusion Array); U.S. Pat. No. 5,087,244; U.S. Pat. No. 5,653,689; Wolinsky, et al. (1990) (Wolinsky Infusion Catheter), “Use of a perforated balloon catheter to deliver concentrated heparin into the wall of the normal canine artery,” J. Am. Coll. Cardiol. 15: 475-481; and Lambert et al., 1993, “Local drug delivery catheters: functional comparison of porous and microporous designs,” Coron. Artery Dis. 4: 469-475; all of which are incorporated herein by reference in their entirety. Use of such catheters for site-directed somatic cell gene therapy is described, e.g., in Mazur, et al., 1994, “Coronary restenosis and gene therapy,” Texas Heart Institute Journal 21: 104-111, hereby expressly incorporated by reference.

Aspects of the invention also concern the ex vivo transfection of ADRCs with a gene encoding a therapeutic polypeptide, and administration of the transfected cells to the mammalian subject. Procedures for seeding a vascular graft with genetically modified endothelial cells are described in, e.g., U. S. Pat. No. 5,785,965, “VEGF gene transfer into endothelial cells for vascular prosthesis,” hereby expressly incorporated by reference in its entirety.

In some embodiments, the administering step comprises implanting a prosthetic or medical device (e.g., intravascular stent) in the mammalian subject, where the stent is coated or impregnated with an adipose-derived cell population that comprises ADRCs. Exemplary materials for constructing valves, stents or grafts coated or seeded with transfected endothelial cells are described in Pavcnik, et al., 2004, “Second-generation percutaneous bioprosthetic valve: a short-term study in sheep,” Eur. J. Endovasc. Surg. 40:1223-1227, and Arts, et al., 2002, “Contaminants from the Transplant Contribute to Intimal Hyperplasia Associated with Microvascular Endothelial Cell Seeding,” Eur. J. Endovasc. Surg. 23:29-38, incorporated herein by reference. See also U.S. patent application Ser. No. 11/317,422, entitled CELL-LOADED PROSTHESIS FOR REGENERATIVE INTRALUMINAL APPLICATIONS, filed Dec. 22, 2005, incorporated herein by reference. For example, in one variation, a synthetic valve that comprises an adipose-derived cell population that comprises ADRCs is sutured to a square stainless steel stent. The square stent has a short barb at each end to provide anchors for the valve during placement, and the submucosa membrane is slit at the diagonal axis of the stent to create the valve opening.

Surfaces of the synthetic valve can be coated with a transfected or non-transfected adipose-derived cell population that comprises ADRCs, e.g., by placing the synthetic valve in an appropriate cell culture medium for 1-3 days prior to implantation to allow for complete coverage of valve surface with the cells.

In another embodiment, the administering step comprises implanting an intravascular stent in the mammalian subject, where the stent is coated or impregnated, as described in literature cited above and reviewed in Lincoff, et al., 1994. A metal or polymeric wire for forming a stent is coated with a composition such as a porous biocompatible polymer or gel that is impregnated with (or can be dipped in or otherwise easily coated immediately prior to use with) a transfected or non-transfected adipose-derived cell population that comprises ADRCs. The wire is coiled, woven, or otherwise formed into a stent suitable for implantation into the lumen of a vessel using conventional materials and techniques, such as intravascular angioplasty catheterization. Exemplary stents that may be improved in this manner are described and depicted in U.S. Pat. Nos. 5,800,507 and 5,697,967 (Medtronic, Inc., describing an intraluminal stent comprising fibrin and an elutable drug capable of providing a treatment of restenosis); U.S. Pat. No. 5,776,184 (Medtronic, Inc., describing a stent with a porous coating comprising a polymer and a therapeutic substance in a solid or solid/solution with the polymer); U.S. Pat. No. 5,799,384 (Medtronic, Inc., describing a flexible, cylindrical, metal stent having a biocompatible polymeric surface to contact a body lumen); and U.S. Pat. Nos. 5,824,048, 5,679,400 and 5,779,729; all of which are hereby expressly incorporated herein by reference in their entirety.

As disclosed herein, the adipose-derived cell population that comprises ADRCs may be provided to the subject, or applied directly to the damaged tissue, or in proximity to the damaged tissue, without further processing or following additional procedures to further purify, modify, stimulate, or otherwise change the cells. For example, the cells obtained from a patient may be provided back to said patient without culturing the cells before administration. In several embodiments, the collection and processing of adipose tissue, as well as, administration of the adipose-derived cell population that comprises ADRCs is performed at a patient's bedside. In a preferred embodiment the cells are extracted from the adipose tissue of the person into whom they are to be implanted, thereby reducing potential complications associated with antigenic and/or immunogenic responses to the transplant. However, the use of cells extracted from another individual is also contemplated.

In accordance with the invention herein disclosed, the adipose tissue-derived cells can be delivered to the patient soon after harvesting the adipose tissue from the patient. For example, the cells may be administered immediately after the processing of the adipose tissue to obtain a composition of adipose tissue-derived stem cells. In one embodiment, the preferred timing of delivery should take place on the order of hours to days after diagnosis of edema or of a procedure likely to place the patient at risk for developing edema. In another embodiment, the harvest and, in certain cases the treatment, can take place in advance of a procedure likely to induce a pancreatic disorder. Ultimately, the timing of delivery will depend upon patient availability and the time required to process the adipose tissue. In another embodiment, the timing for delivery may be relatively longer if the cells to be delivered to the patient are subject to additional modification, purification, stimulation, or other manipulation, as discussed herein. Furthermore, the adipose-derived cell population that comprises ADRCs may be administered multiple times. For example, the cells may be administered continuously over an extended period of time (e.g., hours), or may be administered in multiple injections extended over a period of time. In certain embodiments, an initial administration of the adipose-derived cell population that comprises ADRCs will be administered within about 12 hours after diagnosis of acute pancreatitis or disorder or performance of a procedure likely to induce development of acute pancreatitis, such as at 6 hours, and one or more doses of cells will be administered at 12 hour intervals.

The number of the adipose-derived cell population that comprises ADRCs administered to a patient may be related to the cell yield after adipose tissue processing. In addition, the dose delivered will depend on the route of delivery of the cells to the patient. Fewer cells may be needed when intra-pancreatic delivery systems are employed, as these systems and methods can provide the most direct pathway for treating pancreatic conditions. The cell dose administered to the patient will also be dependent on the amount of adipose tissue harvested and the body mass index of the donor (as a measure of the amount of available adipose tissue). The amount of tissue harvested will also be determined by the extent of the injury or insufficiency. Multiple treatments using multiple tissue harvests or using a single harvest with appropriate storage of cells between applications are within the scope of this invention.

A portion of the total number of cells may be retained for later use or cryopreserved. Portions of the processed adipose tissue may be stored before being administered to a patient. For short term storage (e.g., less than 6 hours) cells may be stored at or below room temperature in a sealed container with or without supplementation with a nutrient solution. Medium term storage (e.g., less than 48 hours) is preferably performed at 2-8° C. in an isosmotic, buffered solution (for example Plasmalyte®) in a container composed of or coated with a material that prevents cell adhesion. Longer term storage is preferably performed by appropriate cryopreservation and storage of cells under conditions that promote retention of cellular function, such as disclosed in PCT App. No. PCT/US02/29207, filed Sep. 13, 2002 and U.S. patent application Ser. No. 60/322,070, filed Sep. 14, 2001, the contents of both of which are hereby expressly incorporated by reference.

In some embodiments, the amount of adipose derived cells (e.g., an enriched, concentrated, isolated, or purified population of the adipose-derived cells comprising ADRCs), which is provided to a subject in need thereof is greater than or equal to about 10,000, 20,000, 30,000, 40,000, 50,000, 60,000, 70,000, 80,000, 90,000, 100,000, 110,000, 120,000, 130,000,140,000, 150,000, 160,000, 170,000, 180,000, 190,000, or 200,000 cells and the amount of ADRCs in said population of adipose derived cells can be greater than or equal to 0.5%-1%, 1-2%, 2%-4%, 4%-6%, 6%-8%, 8%-10%, 10%-20%, 20%-30%, 30%-40%, 40%-50%, 50%-60%, 60%-70%, 70%-80%, 80%-90%, or 90%-100% of the total population of adipose derived cells. The dose can be divided into several smaller doses, e.g., for administering over a period of time or for injection into different parts of the affected tissue, e.g., by local injection. However, this dosage can be adjusted by orders of magnitude to achieve the desired therapeutic effect.

The cells can also be subjected to cell culture on a scaffold material prior to being implanted. Thus, tissue engineered valves, pancreatic vessels, and other structures could be synthesized on natural or synthetic matrices or scaffolds using ADRCs prior to insertion or implantation into the recipient.

Many routes of administration can be suitable for the therapeutics described herein. In some variations, oral, intravenous, intraarterial, and other systemic administrations are used. In some variations, local delivery to an edematous limb or other portion of the body, such as administered subcutaneously at a site of edema, is contemplated.

In some embodiments, direct administration of cells to the site of intended benefit is preferred. This can be achieved by local injection into the tissue, direct injection into a pancreatic structure or pancreatic vessel, through insertion of a suitable cannula, by arterial or venous infusion (including retrograde flow mechanisms) or by other means disclosed herein or known in the art.

The adipose-derived cell population that comprises ADRCs can be applied by several routes including systemic administration by venous or arterial infusion (including retrograde flow infusion) or by direct injection into the pancreatic system. Systemic administration, particularly by peripheral venous access, has the advantage of being minimally invasive relying on the natural transport of cells from the blood to the pancreas. The adipose-derived cell population that comprises ADRCs can be injected in a single bolus, through a slow infusion, or through a staggered series of applications separated by several hours or, provided cells are appropriately stored, several days or weeks. The adipose-derived cell population that comprises ADRCs can also be applied by use of catheterization such that the first pass of cells through the area of interest is enhanced by using balloons. As with peripheral venous access, the adipose-derived cell population that comprises ADRCs may be injected through the catheters in a single bolus or in multiple smaller aliquots. Cells can also be injected into interstitial space.

As previously set forth above, in a preferred embodiment, the adipose-derived cell population that comprises ADRCs is administered directly into the patient. In other words, the active cell population (e.g., the ADRCs, progenitor cells, stem cells and/or combinations thereof) are administered to the patient without being removed from the system or exposed to the external environment of the system before being administered to the patient. Providing a closed system reduces the possibility of contamination of the material being administered to the patient. Thus, processing the adipose tissue in a closed system provides advantages over existing methods because the active cell population is more likely to be sterile. In some embodiments, the only time the adipose-derived cell population that comprises ADRCs are exposed to the external environment, or removed from the system, is when the cells are being withdrawn into an application device and administered to the patient. In other embodiments, the application device can also be part of the closed system. Accordingly, a complete closed system is maintained from removal of the adipose tissue from the subject (e.g., cannula) to introduction to the subject (e.g., application device). Thus, the cells used in these embodiments are may be processed for culturing or cryopreservation and may be administered to a patient without further processing, or may be administered to a patient after being mixed with other tissues, cells, or additives.

In other embodiments, at least a portion of the adipose-derived cell population that comprises ADRCs can be stored for later implantation/infusion. The population may be divided into more than one aliquot or unit such that part of the population of cells is retained for later application while part is applied immediately to the patient. Moderate to long-term storage of all or part of the cells in a cell bank is also within the scope of this invention, as disclosed in U.S. patent application Ser. No. 10/242,094, entitled PRESERVATION OF NON EMBRYONIC CELLS FROM NON HEMATOPOIETIC TISSUES, filed Sep. 12, 2002, which claims the benefit of U.S. application Ser. No. 60/322,070, filed Sep. 14, 2001, the contents of both expressly incorporated herein by reference. At the end of processing, the concentrated cells may be loaded into a delivery device, such as a syringe, for placement into the recipient by any means known to one of ordinary skill in the art. The adipose-derived cell population that comprises ADRCs with or without an additive can be used in several therapeutic methods as described in the following section.

### Therapeutic Methods

Aspects of the invention concern methods of tissue transplantation, methods of modulation inflammation (e.g., in organs), and methods of treatment or reducing or alleviating at least one adverse effect or symptom of a pancreatic condition, disease or disorder, or another disease associated with a misdirected inflammatory response (e.g., acute pancreatitis and acute kidney disease), which entail providing an adipose-derived cell population that comprises ADRCs to a subject that has been identified as one in need of tissue transplantation and/or a subject suffering from an inflammatory disease (e.g., acute pancreatitis). The identification or selection of a subject in need of a tissue transplantation and/or a subject suffering from a inflammatory disease (e.g., acute pancreatitis) can be accomplished by a clinician or physician using evaluation techniques known in the field of medicine. By some approaches, the identification or selection is made using a diagnostic tool and by other approaches the identification or selection is made using clinical or laboratory evaluation, such as observation of symptoms associated with an inflammatory response or disease.

Once a subject in need is identified, the identified subject is provided a therapeutically effective amount of an adipose-derived cell population that comprises ADRCs or a subset of the ADRCs. In some embodiments, a method of treating a patient includes steps of: a) providing a tissue removal system; b) removing adipose tissue from a patient using the tissue removal system, the adipose tissue having a concentration of therapeutic cells; c) processing at least a part of the adipose tissue to obtain a concentration of therapeutic cells other than the concentration of therapeutic cells of the adipose tissue before processing; and d) administering the therapeutic cells to a patient without removing the therapeutic cells from the tissue removal system before they are ready to be administered to the patient using several methods known to one of ordinary skill in the art, including but not limited to, injection into the pancreas, into the blood system, and into tissues and tissue space.

In some embodiments, an adipose-derived cell population that comprises ADRCs used to treat conditions, diseases, and disorders of the pancreatic system. Adipose tissue-derived cells of the invention have properties that can contribute to modulating expansion, repair, or regeneration of pancreatic structures. These properties include, among other things, the ability to synthesize and secrete growth factors that modulate pancreatic cell expansion, as well as the ability to proliferate and differentiate into cells directly participating in the treatment of a pancreatic disorder (e.g., acute pancreatits). The methods and compositions described herein can also be used to modulate re-growth or permeability of pancreatic structures in, for example, organ or tissue transplant patients.

Some of the methods described herein can be used in conjunction with tissue or cell transplantation (e.g., pancreatic islet transplantation) to expedite the formation of pancreatic structures in and around the transplant. Depending on the type of tissue or cells to be transplanted, ADRCs can be provided with the transplant material as a mixture or they can be administered separately by other methods described herein and in the literature, e.g., intravenously, subcutaneously, intraarterially, etc. Additives, e.g., growth factors and immunosuppressive agents, can be co-administered as desired. Administration of the additives as well as the adipose-derived cell population that comprises ADRCs can take place before, during or after the tissue transplantation procedure. The adipose-derived cell population that comprises ADRCs can also be administered via a scaffold, e.g., a resorbable scaffold known in the art.

For pancreatic system disorders resulting from genetic defects, treatment using non-autologous ADRCs cells might prove beneficial. Administration of non-autologous cells using the methods of the invention can be accomplished using methods known in the art and described herein and in the literature.

Cells may be administered to a patient in any setting in which pancreatic function is insufficient or abnormal. In a preferred embodiment, the subject, and the adipose-derived cell population that comprises ADRCs are human. The adipose-derived cell population that comprises ADRCs may be provided in vitro, or in vivo. The cells may be extracted in advance and stored in a cryopreserved fashion or they may be extracted at or around the time of defined need.

### Methods of Screening Compounds

It is contemplated that screening techniques using an adipose-derived cell population that comprises ADRCs will be useful for the identification of compounds that will augment, stimulate or otherwise increase the effects of the ADRCs of the present invention and be useful in the treatment of inflammatory disorders in general (e.g., pancreatitis). It is similarly contemplated that such screening techniques will prove useful in the identification of compounds that will inhibit the ability of ADRCs to modulate the inflammatory response In some embodiments, the present invention is directed to a method for determining the ability of a candidate substance to modulate the growth or activity of, for example, the pancreatic system.

For example, another aspect of the invention concerns methods of identifying compounds that modulate expansion of pancreatic cells (e.g., acinar cells), the formation of pancreatic vessels or the formation of pancreatic tissue. By some approaches, a test compound is contacted with a composition comprising an adipose-derived cell population that ADRCs. Next, the ability of said test compound to modulate expansion of pancreatic cells, the formation of pancreatic vessels or the formation of pancreatic tissue is determined or measured. A candidate compound that increases or decreases the ability of said adipose-derived cell population that comprises ADRCs to modulate expansion of pancreatic cells, the formation of pancreatic vessels or the formation of pancreatic tissue in comparison to control cells not exposed to the candidate compound is then identified. In some embodiments the adipose-derived cell population that comprises ADRCs is identified as a source of ADRCs.

To identify a candidate substance as being capable of promoting or inhibiting the growth of a pancreatic cell network, one could, e.g., measure or determine the presence of growth of ADRCs of the present invention in the absence of the added candidate substance. One could then add the candidate substance to the co-cultured cells and determine the response of the co-culture in the presence of the candidate substance. A candidate substance that modulates the expansion of pancreatic cells in the co-culture is indicative of a candidate substance having the desired activity. In in vivo screening assays, the compound can be administered to a model animal, over a period of time and in various dosages, and an alleviation of the symptoms associated with edema or tumor progression or tumor metastasis monitored. Any improvement in one or more of these symptoms can be indicative of the candidate substance being a useful agent.

As used herein the term “candidate substance” refers to any molecule that may potentially act as a modulator pancreatic cell expansion. Such an agent may be a protein or fragment thereof, a small molecule inhibitor, or even a nucleic acid molecule.

Additionally, one can acquire from commercial sources small molecule libraries that are believed to meet the basic criteria for useful drugs in an effort to identify useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds derived from active, but otherwise undesirable compounds. The application of such libraries to the adipose-derived cell population that comprises ADRCs prepared as described herein is also contemplated.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or manmade compounds.

As described, the ADRCs of the present invention can be cultured according to methods known in the art, and the cultured cells used in drug screening assays. “Effective amounts” of the candidate agent in certain circumstances are those amounts effective to reproducibly produce an alteration in the modulation of expansion of pancreatic cells and/or structures. Significant changes in activity and/or expression will be those that are represented by alterations in activity of, e.g., 30%-40%, and preferably, by changes of at least about 50%, with higher values of course being possible. Aspects of the invention also utilize ADRCs that have not been cultured or are fresh, unadulterated cells obtained by any one or more o fthe approaches described herein.

The following examples are provided to demonstrate particular situations and settings in which this technology may be applied and are not intended to restrict the scope of the invention and the claims included in this disclosure.

### EXAMPLES

### Example 1

No clinical therapy for acute pancreatitis is available to date and no therapeutic cell therapy approaches have been published as well. Many prophylactic treatments though have shown efficacy in reducing pancreatitis disease progression in rodent models. However, few interventional therapies have proven efficacious. As an example showing that adipose-derived regenerative cells (ADRCs) can reduce inflammation and/or the presence or amount of a marker for inflammation, independent studies to evaluate the potential of administration of ADRCs to reduce conditions associated with acute pancreatitis were performed.

Two independent studies (n=180 enrolled; n=137 total reaching endpoint) were performed to investigate the therapeutic potential of ADRCs in a murine model of acute pancreatitis. Acute pancreatitis was induced by IP injection of L-Arginine. Therapy was performed by injection of 1×10⁶ syngeneic ADRCs via the tail-vein within 3 to 5 hours of pancreatitis induction. In the first study, animals were sacrificed 48 hours after pancreatitis induction to obtain serum and tissue samples; while in the second study, animals were sacrificed 72 hours after induction for tissue sampling. The major findings from each study can be summarized as follows:

1. Pancreatitis can be reliably and consistently induced in Balb/C mice by IP injection of 3.5 g/kg body weight L-Arginine.

2. Pancreatitis is evident by elevated serum amylase and lipase concentrations within 24 hours of disease induction, which return to normal after 96 hours. In addition, pancreata display evidence of severe pancreatitis within 48 hours, as assessed by histopathology. Accordingly, by some approaches the identification or selection of subjects in need of a treatment, amelioration, or alleviation of at least one adverse effect or symptom associated with an inflammatory condition or disease involves analysis of any on or more of the aforementioned markers.

3. One million ADRCs significantly reduce histopathological indications of pancreatitis at both 48 and 72 hours, relative to PBS controls. Assessment of severity of injury is based upon acinar necrosis and inflammatory cell infiltrate.

4. In contrast to the results found by histopathology, ADRCs do not attenuate the increase of the indirect biomarkers of pancreatitis, serum amylase or lipase, at 48 hours.

Based on these findings, ADRCs may provide a promising and novel therapeutic approach for treating patients presenting with symptoms of acute pancreatitis.

### I. Experimental Design and Model:

### 1. Design:

Experiment 1

| | | No. of | No. Reaching | Time of |
|---|---|---|---|---|
| Group | Treatment | Animals | Endpoint | Sacrifice |
| 1 | 1 × 10⁶ ADRCs | 52 | 39 | 48 hrs. |
| 2 | PBS | 44 | 40 | 48 hrs. |

Experiment 2

| | | No. of | No. Reaching | Time of |
|---|---|---|---|---|
| Group | Treatment | Animals | Endpoint | Sacrifice |
| 1 | 1 × 10⁶ ADRCs | 40 | 31 | 72 hrs. |
| 2 | PBS | 44 | 27 | 72 hrs. |

### 2. Model:

i. Pancreatitis was induced in 6 week old, Balb/C mice by two IP injections of 3.5 g/kg body weight L-Arginine, one hour apart. Pancreatitis induction was modified from the protocol described in the following manuscript: R. Dawra, et al., Development of a new mouse model of acute pancreatitis induced by administration of L-arginine. Am J Physiol Gastrointest Liver Physiol. 2007 April; 292(4):G1009-18.

ii. One million ADRCs or PBS were delivered by tail-vein injection 3 to 5 hours after pancreatitis induction in a total volume of 0.1 mL.

iii. Animals were sacrificed at 48 (Experiment 1) or 72 (Experiment 2) hours, as listed above. Serum and pancreata were obtained for analysis.

iv. Serum was analyzed or measured for amylase and lipase concentrations. Accordingly, in some contexts, the response of the subjects that received the ADRCs were measured before and after receiving the ADRCs. Such measurements may be optionally applied in some of the methods described herein.

v. Histologic sections of pancreata were scored or evaluated for acinar cell necrosis and perivascular infiltrate to indicate severity of pancreatitis. Two independent investigators scored three randomized high power fields from each pancreas (400× magnification). Scoring strategies are shown in Table 3:

Table 3. Scoring Criteria for Pancreatitis Pathology. The scoring strategy used was based on criteria described in the following manuscript: J, Schmidt, et al., A better model of acute pancreatitis for evaluating therapy. Ann Surg. 1992 January; 215(1):44-56.

Histopathology Scoring Index for Pancreatitis

| Score | |
|---|---|
| | Acinar Cell Necrosis |
| 0 | Absent |
| 1 | Diffuse occurrence of 1-4 necrotic cells/HPF |
| 2 | Diffuse occurrence of 5-10 necrotic cells/HPF |
| 3 | Diffuse occurrence of 11-16 necrotic cells/HPF |
| 4 | >16 necrotic cells/HPF (Extensive confluent necrosis) |
| | Perivascular Cell Infiltrate |
| 0 | 0-3 intra-lobular or perivascular leukocytes/HPF |
| 1 | 4-6 intra-lobular or perivascular leukocytes/HPF |
| 2 | 7-9 intra-lobular or perivascular leukocytes/HPF |
| 3 | 10-15 intra-lobular or perivascular leukocytes/HPF |
| 4 | >15 leukocytes/HPF or confluent microabscesses |

### II. Results:

1. Healthy pancreata are shown in FIG. 16, as a normal control.

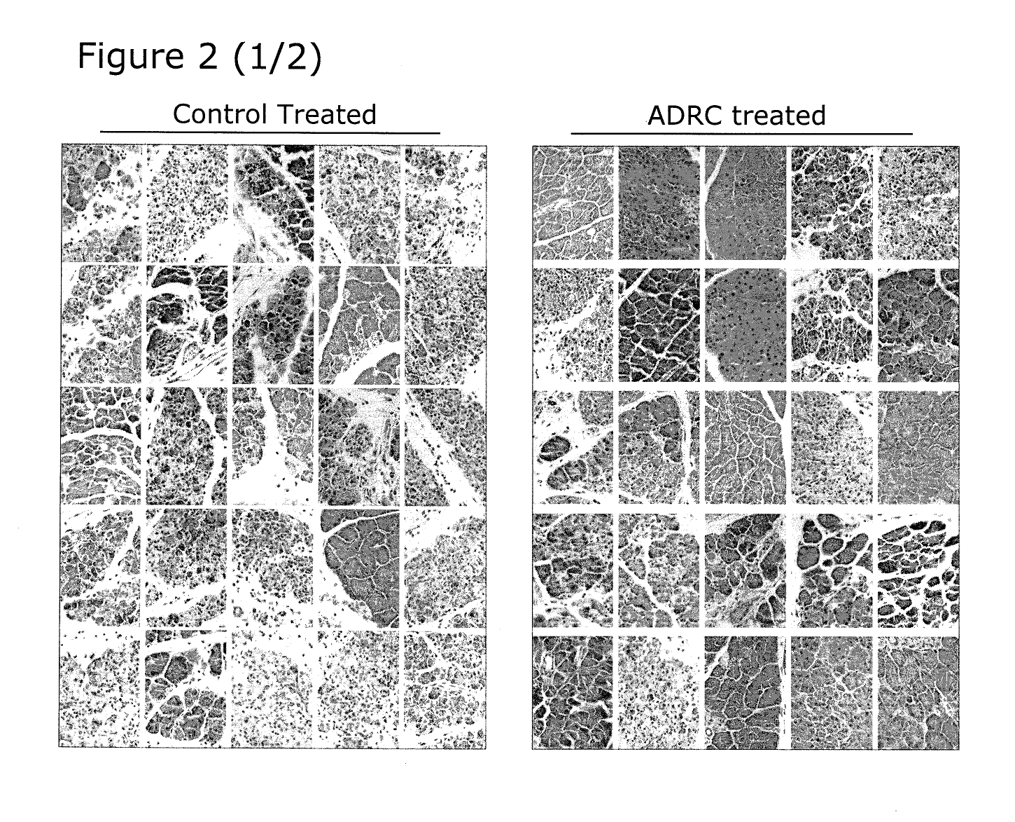

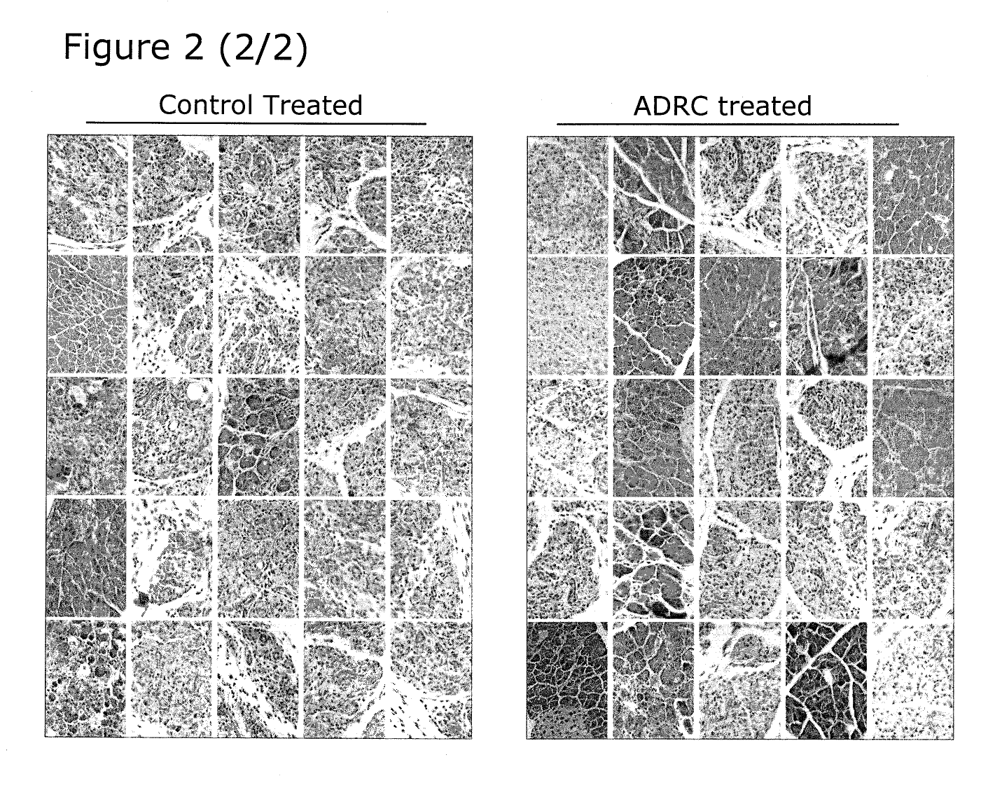

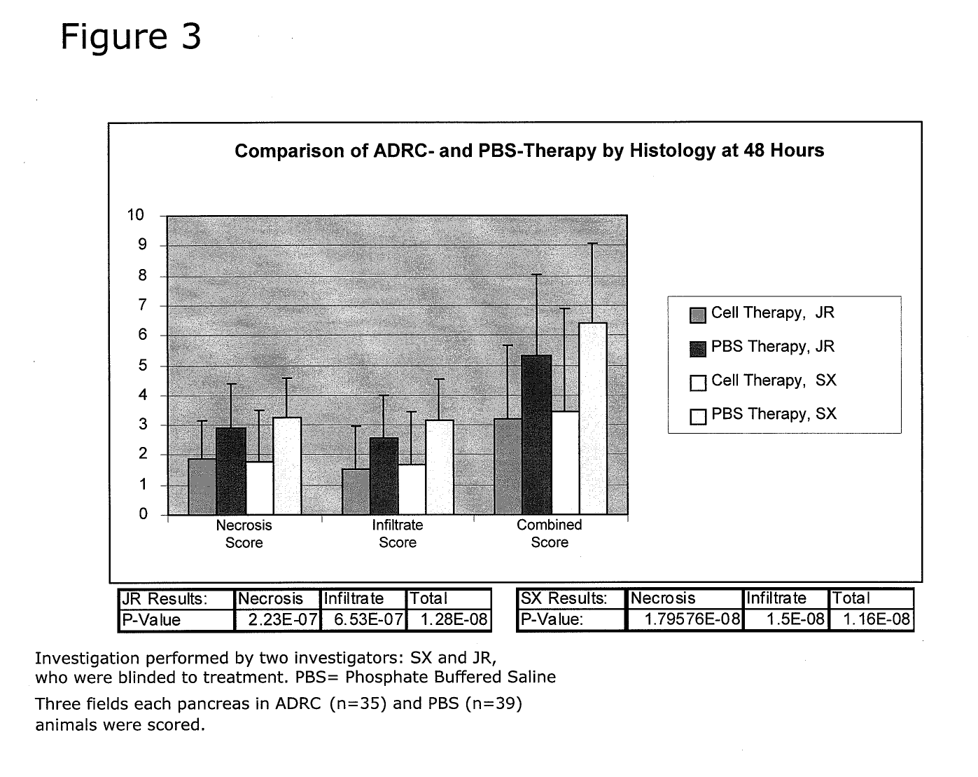

2. Administration of L-Arginine induces acute pancreatitis. By 48 hours, pancreata display histopathological evidence of severe pancreatitis, including acinar cell necrosis and perivascular infiltrate (FIG. 2).

3. ADRC-therapy results in reduced pathology scores of pancreatitis compared to PBS controls at both 48 hours (n=35 for ADRC-treatment, n=39 for control) and 72 hours (n=31 for ADRC-treatment, n=27 for control; see FIGS. 17, 18, and 19). Two independent investigators, scoring randomized fields of pancreata, both demonstrated significant decreases in pathology indices for acinar necrosis and perivascular infiltrate. Scoring strategies are shown in Table 3.

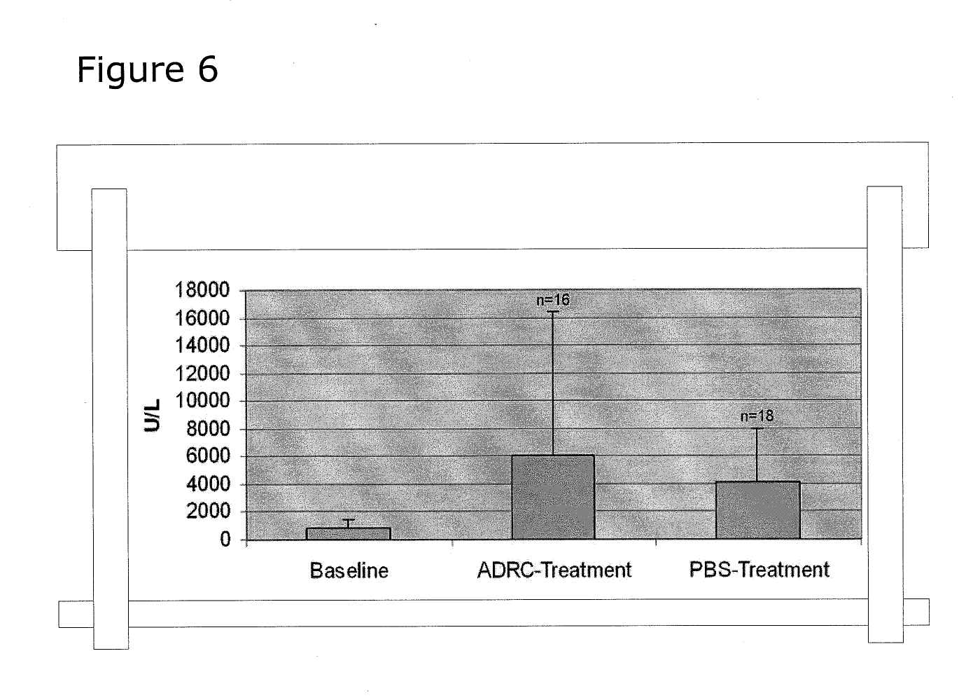

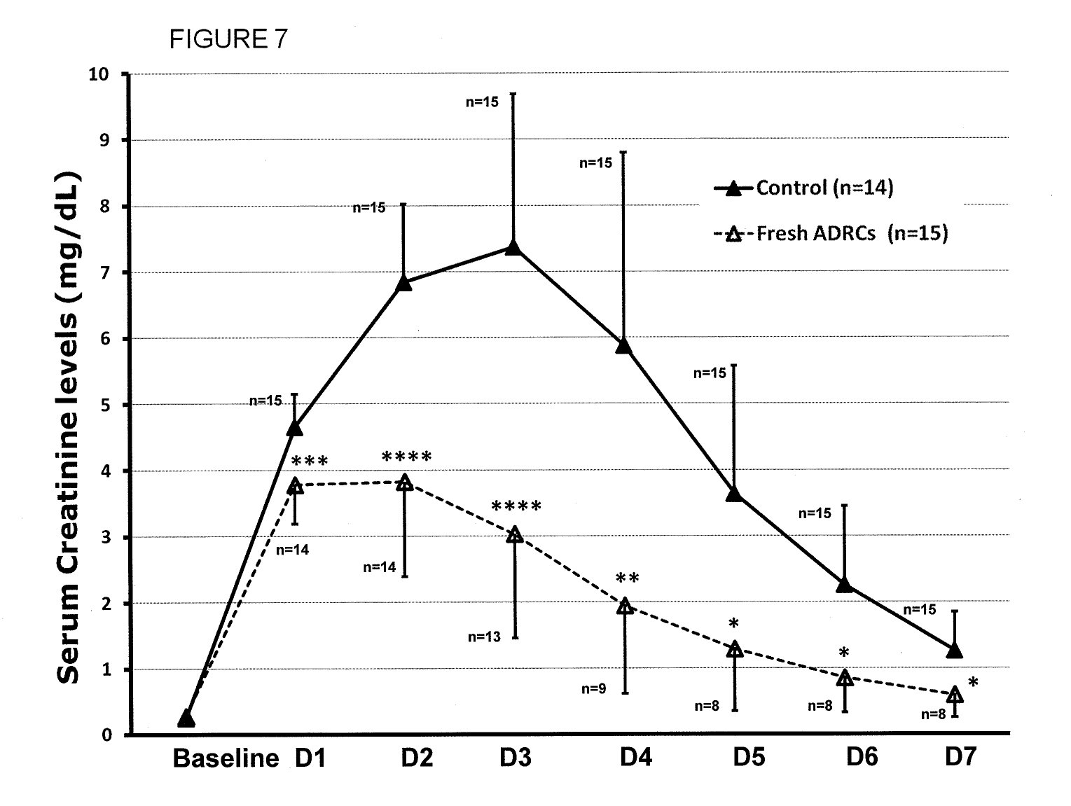

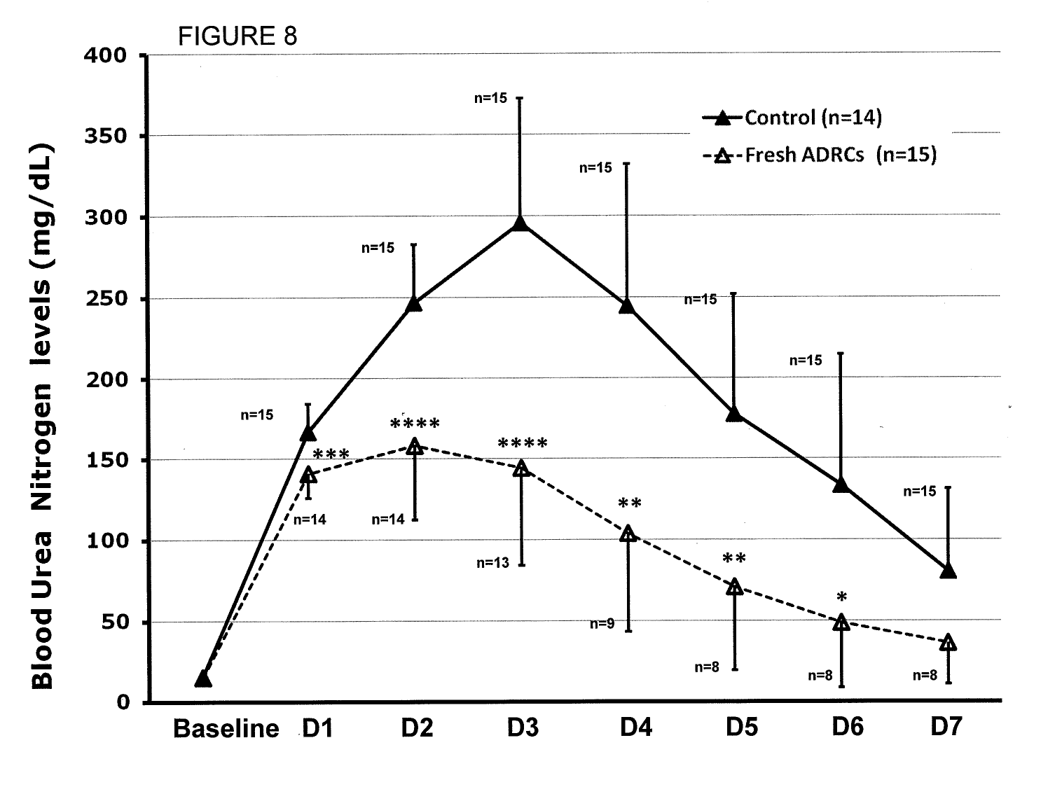

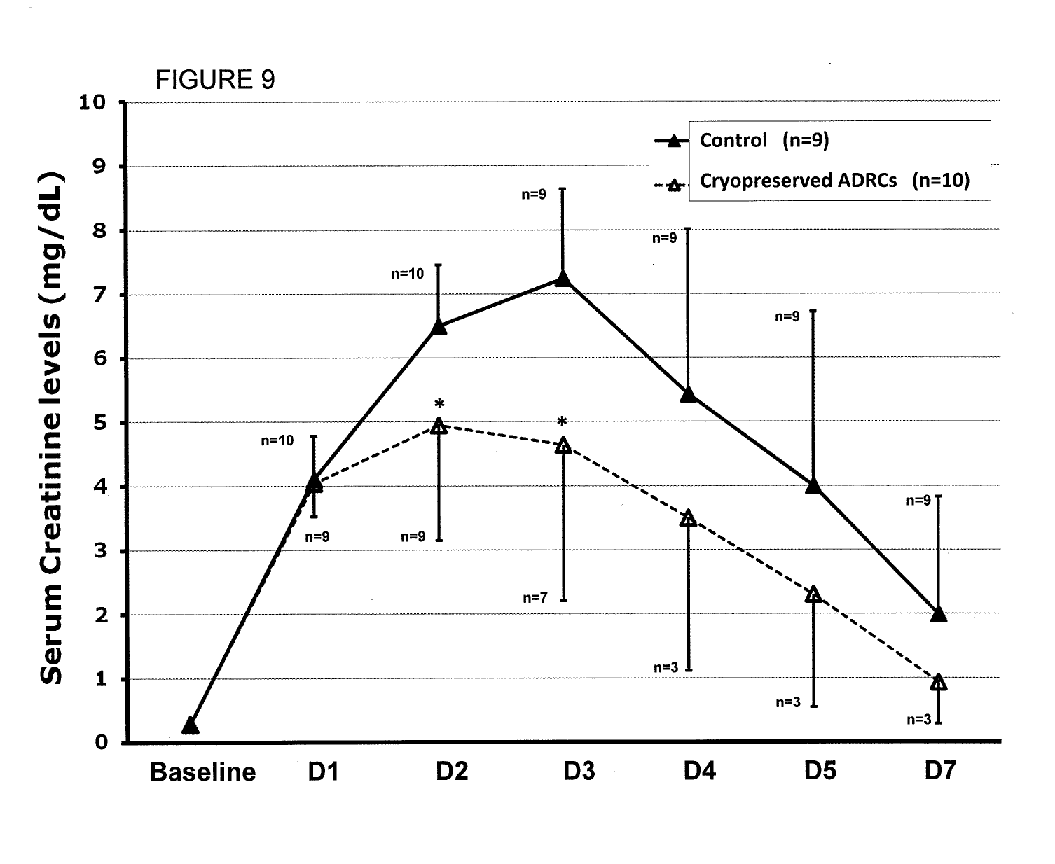

4. Serum concentrations of both amylase and lipase increase 3.9- and 19.6-fold, respectively, at 48 hours. However, compared to control animals, ADRCs did not reduce serum amylase concentrations (n=33 for ADRC-treatment, n=38 for control; FIG. 20) or lipase concentrations (n=16 ADRC-treatment, n=18 for control; FIG. 6) after 48 hours of treatment (measured in samples of Experiment 2 only).

5. Serum enzyme levels and histopathology scores are not predictive of one another.

### III. Conclusions:

1. Based on histology scoring, ADRCs significantly reduce the damage incurred in the pancreatic acinar cells as well as the inflammatory infiltrate due to L-Arginine induced pancreatitis. Accordingly, it was discovered that ADRCs can reduce inflammation and/or the presence or amount of a marker for inflammation.

2. ADRCs do not influence serum amylase and lipase levels, which is consistent with clinical findings that Amylase/Lipase do not predict outcome of pancreatitis.

### Example 2

As a second example showing that adipose-derived regenerative cells (ADRCs) can reduce inflammation and/or the presence or amount of a marker for inflammation, independent studies to evaluate the potential of administration of ADRCs to reduce conditions associated with acute kidney injury (AKI) were performed. The following example demonstrates that Adipose Tissue-Derived Stem and Regenerative Cells (ADRC) significantly decrease mortality and increase renal function in a preclinical model of acute kidney injury (AKI) induced by ischemia/reperfusion. Administration of ADRCs modified key inflammatory events like the expression of the pro-inflammatory cytokines Chemokine (C-X-C motif) ligand 2 (CXCL2) and Interleukin-6 (IL-6) as well as the infiltration of macrophages. The result is a significant reduction in organ damage as shown by reduced tubular necrosis/apoptosis and an increase in the proliferation of tubular cells, thus improving functional and structural recovery and overall survival. Collectively, this work suggests that both freshly isolated and cryopreserved ADRCs significantly accelerate renal repair and preserve renal function, offering a potential therapeutic approach in renal reparative medicine and inflammatory diseases in general.

### Animals and Induction of the Ischemia Reperfusion Injury

Adult male inbred Fisher 344 rats (100-200 g) and surgically modified (common carotid artery cannulated) male adult Fisher 344 rats (200-300 g) were used as syngeneic ADRC donors and recipients, respectively (Taconic, Hudson, N.Y.). Animals were kept under temperature-controlled conditions of 12-h light/dark cycle, with water and food ad libitum. The animal study protocol was in accordance with National Institutes of Health (NIH) Guide for the Care and Use of Laboratory Animals (U.S. Department of Health and Human Services Public Health Services, NIH, NIH Publication No. 86-23, 1985) and approved by the in-house Animal Care and Use Committee. Renal ischemia-reperfusion (I-R) was generated according to Togel and colleagues with minor modifications. Briefly surgically modified male adult Fisher 344 rats (200 g-300 g) were anesthetized with a mixture of 2.5% Isoflurane in oxygen. Core body temperature was monitored and maintained at 37±1° C. throughout the surgical procedure. Both kidneys were exposed and the renal vascular pedicles (artery and vein) were exposed by blunt dissection and then clamped for 38 minutes using nontraumatic microaneurysm clamps (Fine Science Tools, Foster City, Calif.). Effectiveness of clamping was confirmed visually by darkening of the kidneys. Reperfusion was allowed by release of the clamps and confirmed by color change of the kidneys (from dark red to pink). The incisions were sutured and post-operation animals were recovered in a warm environment.

### Experimental Groups

Two separate experiments were performed to evaluate the efficacy of freshly isolated ADRCs and cryopreserved ADRCs. To assess potential renoprotection of fresh ADRCs, a total of fifty-seven (57) rats were subjected to bilateral renal arterial and venous clamping, as described above. Approximately twenty (20) minutes after reperfusion, animals received an intra-arterial infusion of 200 μl of either vehicle control (Phosphate Buffered Saline, PBS) or 5×10⁶ ADRCs. To evaluate efficacy of freshly isolated ADRCs, twenty-nine (29) rats (ADRC: n=15, Control: n=14) had serum creatinine (sCr) and blood urea nitrogen (BUN) levels monitored prior to surgery (baseline) and daily for 1 week after AKI. Also, survival rates were recorded daily. For evaluation of the mechanism of action, twenty-eight (28) rats underwent the surgical procedure and treatment and were euthanized at 5 minutes (ADRC: n=2), 2 hours (ADRC: n=6, Control: n=3), 24 hours (ADRC: n=6, Control: n=3) or 72 hours post-surgery (ADRC: n=5, Control: n=3). In ten (10) of the ADRC-treated rats (n=3 each at the 2 and 24 hour sacrifice timepoints, n=2 at 5 minutes and 72 hour sacrifice timepoints) the cells were DiI-labeled for tracking ADRC engraftment within the kidneys. The remaining rats (ADRC=3, Control=3 at each timepoint) that were sacrificed at 2 and 24 hours post-AKI, had one kidney snap-frozen in liquid nitrogen and stored at −80° C. (for RNA isolation), while the other kidney was used for histology. All six (6) rats (ADRC=3, Control=3) sacrificed at 72 hours post-AKI were evaluated histologically.

In the second experiment, we evaluated cryopreserved ADRCs in the AKI model described above. For this assessment, nineteen (19) rats were enrolled and received an intra-arterial infusion of 200 μl of either vehicle (Control, n=9) or 5×10⁶ recovered ADRCs from cryopreservation (n=10). sCr was evaluated on days 1-5 and 7 after AKI, with daily monitoring of animal survival.

### Isolation of ADRCs

ADRCs were isolated from adult male Fisher 344 rats (100-200 g, Taconic, Hudson, N.Y.) as previously described with minor modifications. Briefly, inguinal subcutaneous adipose tissue was removed from donor rats and minced. Adipose tissue was digested with 0.09% collagenase (Sigma-Aldrich, St. Louis, Mo.) for 45 minutes at 37° C. The ADRC fraction was separated by centrifugation at 600 g for 5 min and passed through 100 μm and 40 μm Falcon™ cell strainers (BD Biosciences, San Jose, Calif.), sequentially. All cells were washed in PBS and incubated with Intravase™ (Cytori Therapeutics, San Diego, Calif.) for 10 minutes followed by more washing in PBS. ADRCs were then counted and resuspended at 25×10⁶ cells/ml in PBS for cell infusion.

### ADRC Labeling for Tracking

After cell isolation, fresh ADRCs were labeled with Vybrant® DiI cell-labeling solution (Invitrogen, Carlsbad, Calif.) according to the manufacturer's instruction with some modifications. Briefly, ADRCs were incubated with cell-labeling solution for 15 min at 37° C. and for another 15 min at 4° C. The labeled cells were rinsed in PBS twice to remove all unbound dye, counted and resuspended at 25×10⁶ cells/ml in PBS and kept on ice until infusion.

### ADRC Cryopreservation

ADRCs were isolated and frozen in 10% syngeneic Fisher 344 rat serum, 10% Dimethyl sulfoxide (DMSO) in Lactated Ringers solution using a Cryogenic Control Rate Freezer 2000 (MVE Biological Systems, Marietta, Ga.). Cooling was performed at −1° C./min from 4° C. to −50° C., then at −10° C./min to −90° C. Once frozen, cells were stored in the vapor phase of liquid nitrogen for at least 48 hours. Prior to infusion, cells were thawed rapidly and resuspended in 10 times volume of PBS. The cells were centrifuged at 400 g for 10 minutes and then washed twice in PBS. Recovered ADRCs were then resuspended to 25×10⁶ cells/ml.

### Flow Cytometric Analysis ADRCs

Flow cytometric studies were performed on fresh and cryopreserved cells. Cells from pooled adipose tissue of animals were first resuspended in staining buffer and then incubated for 10 minutes at 4° C. with blocking antibodies to prevent nonspecific binding to Fc receptors. Cells were then incubated in various combinations for 30 minutes at 4° C. with the following antibodies (all from BD Pharmingen): CD45FITC (554878), CD31PE (555027), CD11bPE (554262), CD73PE (551124) and CD9OPE (551401). Cells were washed twice with cold Dulbecco's Phosphate Buffered Saline (D-PBS) and fixed using a commercial solution (BD #349202). Acquisition and analysis of the cells were performed on a FACSAria using FACSDiva software (BD Biosciences, San Jose, Calif.). Appropriate FMO (fluorescence-minus-one) controls were used in all experiments.

### Assessment of Renal Function

Renal function was monitored by measuring serum creatinine (sCr) and/or blood urea nitrogen (BUN) and blood was withdrawn from an intra-arterial cannula and sCr concentration was determined using i-STAT 1 hand analyzer (Abbott Laboratories, Abbott Park, Ill.). BUN was measured enzymatically at IDEXX laboratories (Westbrook, Me.).

### Histology and Immunohistochemistry

Kidneys were removed and bisected at the designated time points, fixed for 24 hours in 10% buffered formalin then embedded in paraffin. Tissue sections (10 μm thick) from post-AKI day 3 kidneys (n=3 for each group) were rehydrated and stained with Hematoxylin & Eosin. Tubular necrosis and intratubular cast formation were semiquantitatively scored to assess tubular injuries. Tubular necrosis was scored using a previously defined, semi-quantitative score³⁶ with some modifications. The percentage of tubules in the corticomedullary junction that displayed cell necrosis, loss of the brush border and shedding of epithelial cells into the tubular lumen was calculated in six corticomedullary fields (magnification, 400×) with the following criteria: 0=no damage; 1=1-25% of the corticomedullary junction injured; 2=25-50%; 3=50-75%; 4=more than 75%.

The extent of intratubular cast formation was also evaluated and scored in six corticomedullary regions using a previously defined system with some modifications³⁷ (magnification, 200×): 0=no intratubular cast formation within each high-powered field (HPF); 1=1 to 25% of tubules contained cast; 2=25% to 50% tubules contained cast; 3=50% to 75% tubules contained cast; 4=75% to 100% tubules contained cast.

Another set of the paraffin embedded sections (5 μm thick) from the post AKI day 3 rats were stained with a mouse anti-rat CD-68 antibody (Serotec, Oxford, UK) according to standard protocols. Briefly, sections were deparaffinized, rehydrated, and underwent trypsin (0.1%) based proteolytic antigen retrieval. Non-specific binding was blocked using 10% normal donkey serum (Jackson ImmunoResearch Laboratories, West Grove, Pa.) for 20 minute at room temperature prior to incubation with anti-rat CD-68 primary antibody for one hour at room temperature. Negative controls used normal mouse IgG (BD Biosciences, Md., USA). After multiple PBS washes, the sections were incubated sequentially with biotinylated donkey anti-mouse IgG secondary antibody (Jackson ImmunoResearch Laboratories, West Grove, Pa.). The VECTASTAIN ABC Kit (Vector Laboratories, Burlingame, Calif.) was used for subsequent steps followed by development of staining with diaminobenzidine (DAB; Dako North America, Carpinteria, Calif.) and sections were counterstained with hematoxylin. The number of DAB-positive cells were counted in six corticomedullary fields (magnification, 200×) to quantify the macrophage infiltration.

For preparation of the frozen sections, kidneys were fixed in 4% paraformaldehyde at 4° C. for 24 hours and then cryoprotected in 30% sucrose in PBS. Embedding was performed in an optimal cutting temperature compound (OCT; Tissue-Tek, Torrance, Calif.), and samples were stored at −80° C. Frozen samples were cut into 8 μm-thick sections (Cryomicrotom CM 3050S, Leica Microsystems, Bannockburn, Ill.) and the sections were dehydrated and air-dried. To detect cell proliferation, kidneys were collected at 24 hours after AKI and sections were stained with rabbit anti-rat Ki67 (Thermo Scientific, Fremont, Calif.), followed by goat anti-rabbit IgG AlexaFluor 568 (Invitrogen, Carlsbad, Calif.). Sections were washed in PBS, rinsed in water then mounted with Vectashield mounting medium with DAPI to counter-stain the nuclei (Vector Laboratories, Burlingame, Calif.). Nonspecific binding was blocked by a 20 min incubation with 10% normal goat serum (Jackson ImmunoResearch Laboratories, West Grove, Pa.) under room temperature. Control sections were prepared by omitting the primary antibody. The number of Ki-67 positive cells were counted in six corticomedullary fields (magnification, 200×) to assess proliferation. All histological analysis was performed in a blinded fashion.

For the assessment of in vivo cell tacking, frozen sections were mounted with Vectashield mounting medium with DAPI to counter-stain the nuclei (Vector Laboratories, Burlingame, Calif.). All the images (bright field/fluorescence) were visualized using an Olympus BX61 fluorescence microscope (Olympus Optical, Tokyo, Japan). Images were captured by Retiga-EXi digital camera (Q Image, Surrey, BC, Canada) attached to the microscope and images were acquired using Simple PCI software (Hamamatsu Corporation, Sewickley, Pa.).

### RNA Extraction and Real-Time PCR

Kidneys were minced and incubated in RNAlater (Ambion, Austin, Tex.) overnight at 4° C. and then transferred to −80° C. The kidney tissue was homogenized and total RNA was isolated using a Qiagen RNAeasy Midi kit (Qiagen, Valencia, Calif.) according to the manufacturer's instructions. RNA integrity was assessed by 2% agarose gel electrophoresis and its purity was verified by spectrophotometry. Real-time quantitative PCR was carried out on an ABI Prism AB 7500 (Applied Biosytems, Foster City, Calif.) using a TaqMan Universal PCR Master Mix Kit (Applied Biosytems, Foster City, Calif.). Briefly, 1 μg of DNase-treated total RNA was reverse transcribed using Invitrogen SuperScript III Reverse Transcriptase (Invitrogen, Carlsbad, Calif.). Then samples were heated for 10 minutes at 95° C., followed by 40 cycles of denaturation at 95° C. for 15 seconds, and annealing and elongation at 60° C. for 1 minute. Specific TaqMan primers and probes for Interleukine-6 (IL-6, Rn99999011_ml) and Chemokine (C-X-C motif) ligand 2 (Cxcl2, Rn00586403_ml) were obtained from Applied BioSystems (Foster City, Calif.). The housekeeping gene, Rat 13-actin, (Applied Biosytems, Foster City, Calif.) was used to normalize the target gene threshold cycle (Ct) values. Triplicate amplifications were performed for each sample. The AACt method was used for each gene to calculate fold-change differences in gene expression between ADRC-treated and control (PBS-treated) animals.

### Statistical Analysis

All data were expressed as Mean±Standard Deviation (SD). Unpaired, two tailed Student's t test and ANOVA were used to assess differences between data means as appropriate. Survival data was analyzed using the Kaplan-Meier method and groups were compared with the Log-rank test. Histopathology scoring data were analyzed using non-parametric Wilcoxon/Kruskal-Wallis' test. All statistical analysis was performed using the JMP-7 software package (SAS Institute, Cary, N.C.). A P-value at or below 0.05 was considered significant.

### Characteristics of ADRCs

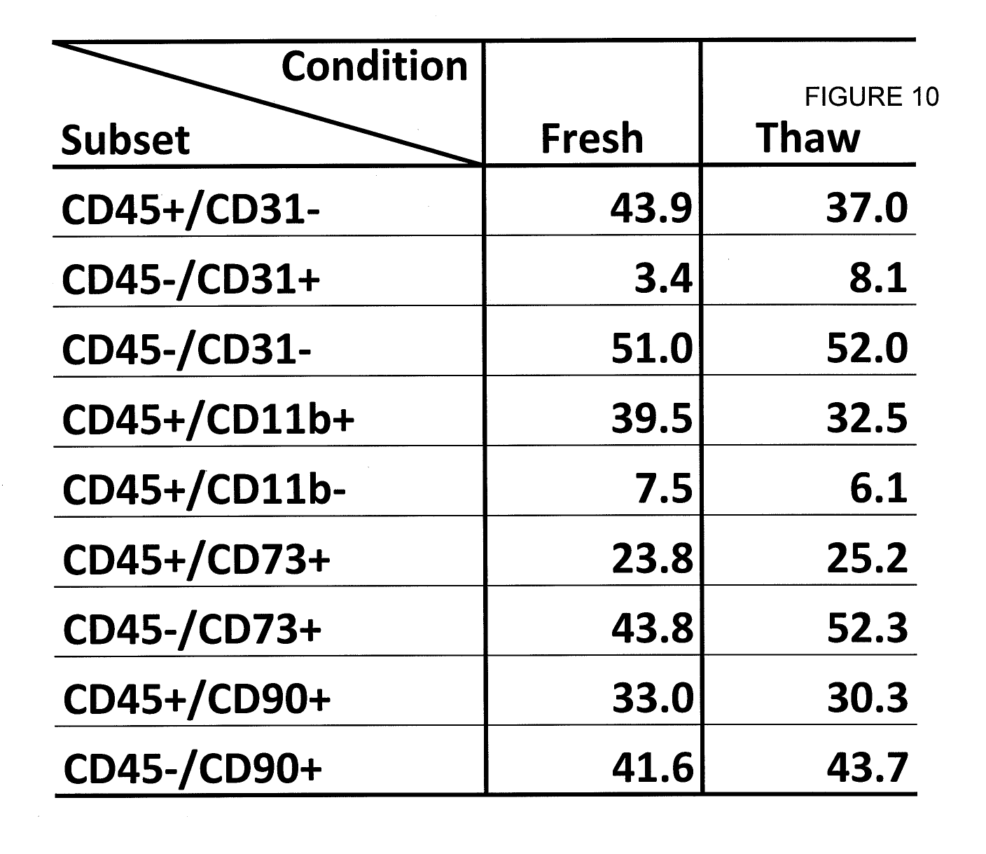

The cell yield of freshly isolated ADRCs was 1.24±0.39×10⁶ cells per gram of the pooled rat adipose tissue (n=9). Flow cytometric evaluation of these cells showed that they comprised an average of approximately 44% CD45⁺ cells (blood and tissue-derived leucocytes), 3.4% endothelial cells (CD45⁻/CD31⁺), and 51% cells that expressed neither CD45 or CD31. CD11b⁺ cells (neutrophils, monocytes, and tissue macrophages) comprised the majority of CD45⁺ cells while CD73 and CD90 were expressed by the majority of CD45⁻ cells. These populations were retained following cryopreservation (FIG. 10) although there was a slight reduction in the frequency of CD45⁺/CD11b⁺ cells and a corresponding increase in CD45⁻/CD31⁺ cells consistent with greater sensitivity of neutrophils to freeze/thawing.

### ADRC Treatment Abolished AKI-Induced Mortality

This ischemia-reperfusion (I-R) model of AKI resulted in a high mortality (43%-67%) in rats that received control (Phosphate Buffered Saline, PBS) treatment with the peak in mortality seen between 3 and 5 days after injury. The infusion of freshly isolated ADRCs rescued all animals from acute mortality resulting in a significant difference in survival (100% vs. 57%, P=0.005, Log rank test). Similarly, animals treated with cryopreserved ADRCs also showed a significant improvement in survival post-AKI. Only 33% of the control (PBS-treated) rats survived compared to 90% of the ADRC-treated group (P=0.019, Log rank test).

### ADRC Administration Improved Renal Functional Recovery

Serum creatinine (sCr) and blood urea nitrogen (BUN) levels were analyzed as surrogate markers of renal function. The mean baseline (prior to I-R injury) sCr and BUN values of all rats were similar. As expected, sCr and BUN levels were significantly elevated at 1 day post-AKI in all animals (FIGS. 2B & 2C). However, while the sCr and BUN levels in control (PBS-treated) rats continued to rise through day 3, rats treated with ADRCs showed a significantly accelerated recovery with overall lower sCr and BUN values. It is important to consider that while the control rats also demonstrated recovery of sCr and BUN levels, albeit delayed, this data is skewed as it only includes surviving animals leading to fewer animals for comparison after day 3. The largest difference in sCr and BUN levels was observed on post-AKI day 3 (sCr: 3.03±1.58 mg/dl vs. 7.37±2.32 mg/dl, P<0.0001; BUN: 144.6±60.48 mg/dl vs. 296±76.74 mg/dl, P<0.0001, for ADRC and controls, respectively). Cryopreserved ADRCs showed a similar response in sCr values with the largest difference seen on day 3 (4.64±2.43 mg/dl vs. 7.24±1.40 mg/dl, P<0.05, ADRC vs. Control).

### ADRCs Infusion Dramatically Attenuated Acute Tubular Necrosis and Intratubular Cast Formation

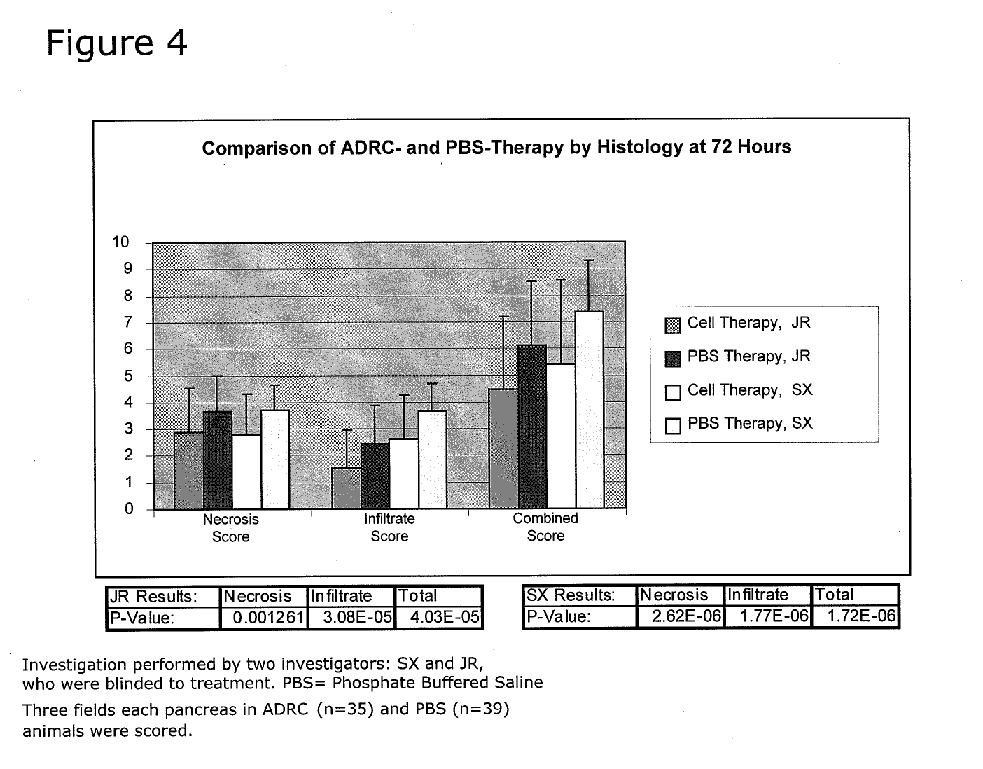

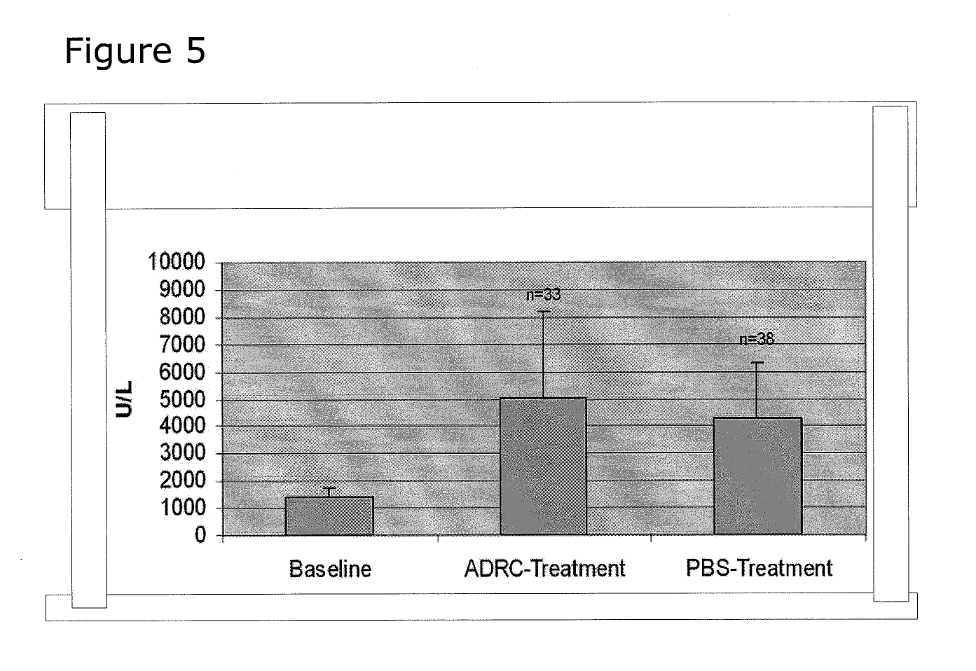

Histologic analysis revealed I-R injury leads directly to acute tubular necrosis and cast formation, both of which are rarely found in healthy kidneys (FIG. 4). Examination of kidneys obtained from control (PBS-treated) animals at 72 hours after AKI (the time point when the majority of animals are still alive in the control group) demonstrated a significant degree of renal injury and exhibited degeneration of tubular structures including severe tubular necrosis, loss of brush border and tubular dilatation. Quantification of this injury/degeneration using the tubular necrosis score demonstrates the extent of tubular damage (3.50±0.79 vs. 0.11±0.32; P<0.0001 control vs. normal kidney). The beneficial effects of ADRCs on restoring renal function were corroborated by histological evidence, showing that ADRC administration markedly reduced the severity of acute tubular necrosis compared with kidneys obtained from control (PBS-treated) animals at 72 hours post-AKI (0.39±0.50 vs. 3.50±0.79; P<0.0001 ADRC vs. control animals).

We also show that there was extensive cast formation in the cortex and outer medullar region in control rats, which is almost absent in healthy kidneys. There was a significant reduction in cast formation in those rats treated with ADRCs compared to control rats on post-AKI day 3 (0.75±0.50 vs. 3.97±0.17; P<0.0001 ADRC vs. control animals).

### ADRC Engraftment in the Injured Kidney

ADRCs were detected by the presence of DiI-positive cells in the glomeruli as early as 5 minutes after infusion. This staining was still evident at 2 h but declined thereafter with a reduced intensity at 24 hours. However, staining was still detectable within glomeruli at 72 hours after ADRCs administration.

### ADRCs Promoted Tubular Epithelial Cell Proliferation

Tubular epithelial cell proliferation was assessed by Ki-67 staining. Abundant Ki-67 positive staining was found in the distal and proximal tubular region of kidneys treated with ADRC at day 1. Ki-67 positive cells were rare in control (PBS-treated) animals (0±1 Ki-67 positive cells/visual field vs. 8±6 Ki-67 positive cells/visual field in control and ADRC animals, respectively; P<0.0001).

### ADRCs Therapy Significantly Decreased CD68-Positive Cell Infiltration

Control (PBS-treated) animals exhibited prominent infiltration of CD68-positive cells (a macrophage marker in the tubulointerstitial compartment of the renal cortex and outer medulla in kidneys at 3 days post-AKI, consistent with the acute inflammatory response following I-R injury. In contrast, treatment with ADRCs resulted in a 25-fold decrease in CD68-positive cell infiltration (6±7 vs. 154±75 cells/ visual field in ADRC and control animals, respectively; P<0.0001).

### ADRCs Treatment Down Regulated Inflammatory Related Gene Expression

In order to further evaluate the reasons for reduced CD68-positive cell infiltration after ADRC therapy, the expression of Chemokine (C-X-C motif) ligand 2 (CXCL2) and Interleukin-6 (IL-6) was assessed at 2 and 24 h post-AKI. We detected a significant down-regulation of both CXCL2 and IL-6 mRNA expression at 24 hours after AKI in the ADRC treated animals compared with those receiving PBS.

What is claimed is:

1. A method for reducing an inflammatory response in a mammal in need thereof comprising providing to said mammal an amount of a concentrated population of adipose derived regenerative cells sufficient to reduce the amount of a marker for inflammation in said mammal.

2. The method of claim 1, wherein said marker for inflammation is IL-3, IL-6, IL-8, IL-12, a Chemokine (C-X-C motif) ligand 2 (CXCL2), or macrophage infiltration.

3. The method of claim 1, wherein the adipose derived regenerative cells are CD14 positive.

4. The method of claim 1, wherein the adipose derived regenerative cells are CD11b positive.

5. The method of claim 1, wherein the mammal has a pancreatic disorder or a kidney disorder.

6. Use of a therapeutically effective amount of a concentrated population of adipose derived regenerative cells to prepare a medicament for the reduction of inflammation, wherein said concentrated population of cells is to be administered to a patient in need thereof without culturing the cells before administering them to the patient.

7. The use of claim 6, wherein the adipose derived regenerative cells are CD14 positive.

8. The use of claim 6, wherein the adipose derived regenerative cells are CD11b positive.

9. The use of claim 6, wherein the mammal has a pancreatic disorder or a kidney disorder.

10. A method of ameliorating pancreatitis or a condition associated therewith comprising:
selecting a patient that has pancreatitis;
administering to said patient a therapeutically effective amount of a concentrated population of adipose derived regenerative cells, wherein said concentrated population of cells is administered to said patient without culturing the cells before administration; and optionally, measuring the response of said patient before and/or after receiving said concentrated population of adipose-derived regenerative cells.

11. The method of claim 10, wherein a marker for inflammation selected from the group consisting of IL-3, IL-6, IL-8, IL-12, a Chemokine (C-X-C motif) ligand 2 (CXCL2), and macrophage infiltration is measured before and/or after receiving said concentrated population of adipose-derived regenerative cells.

12. The method of claim 10, wherein the adipose derived regenerative cells are CD14 positive.

13. The method of claim 10, wherein the adipose derived regenerative cells are CD11b positive.

14. Use of a therapeutically effective amount of a concentrated population of adipose derived regenerative cells to prepare a medicament for the treatment of pancreatitis, wherein said concentrated population of cells is to be administered to a patient in need thereof without culturing the cells before administering them to the patient.

15. The use of claim 14, wherein the adipose derived regenerative cells are CD14 positive or CD11b positive.
